(19) United States
(12) Patent Application Publication
(10) Pub. No.: US 2026/0284392 A1
(43) Pub. Date: Sep. 24, 2026

(54) TREATMENT OF SPINAL MUSCULAR ATROPHY

(71) Applicants: University of Pittsburgh - Of the Commonwealth System of Higher Education, Pittsburgh, PA (US); Genentech, Inc., South San Francisco, CA (US)

(72) Inventors: Marco CAPOGROSSO, Pittsburgh, PA (US); Mikael John Lars ELIASSON, South San Francisco, CA (US); Genis PRAT ORTEGA, Pittsburgh, PA (US)

(21) Appl. No.: 18/873,532

(22) PCT Filed: Jun. 14, 2023
(86) PCT No.: PCT/US2023/068464

§ 371 Date: Dec. 10, 2024

Related U.S. Application Data
(60) Provisional application No. 63/352,599, filed on Jun. 15, 2022.
(60) Provisional application No. 63/450,901, filed on Mar. 8, 2023.

(51) Int. Cl. A61N 1/36; A61N 1/05

(52) U.S. Cl. CPC A61N 1/36057; A61N 1/0553; A61N 1/0556; A61N 1/36062; A61N 1/36157; A61N 1/36171; A61N 1/36175; A61N 1/36178; A61N 1/36182; A61N 1/0558

(57) ABSTRACT

Disclosed herein are methods for treating spinal muscular atrophy in a subject. Particular methods comprise applying a therapeutically effective amount of an electrical stimulus to sensory neurons innervating a body region of the subject with a motor impairment due to the spinal muscle atrophy, wherein application of the electrical stimulus treats the motor impairment due to spinal muscular atrophy in the subject.

### CROSS-REFERENCE TO RELATED APPLICATIONS

This application claims the benefit of U.S. Provisional Application No. 63/352,599, filed Jun. 15, 2022, and U.S. Provisional Application No. 63/450,901, filed Mar. 8, 2023, the entire contents of which are hereby incorporated by reference herein.

### FIELD

The present disclosure relates to methods of treating Spinal Muscular Atrophy (SMA) in a subject by stimulation of sensory afferents in the subject.

### BACKGROUND

SMA is a neurodegenerative disease triggered by a genetic mutation in the survival motoneuron 1 (SMN1) gene (Lefebvre et al., 1995. Cell 80(1):155-165). Affected motoneurons (MNs) in SMA patients are less-capable of producing sustained firing and can degrade over time, leading to MN death. Strikingly, even though the SMN1 gene is ubiquitously expressed in all MNs, not all muscles are affected. SMA specially affects lower limbs and, in the more severe cases, respiratory function. Experiments in mouse models have shown that the insufficient expression of SMN protein first produces the dysfunction and, in the late stage of the disease, the death of MNs (Le et al., 2005. Human Molecular Genetics 14 (6): 845-57; Avila et al., 2007. J Clinical Investigation. 117(3):659-671). In other words, many, if not all, MNs are non-functioning or dead in an SMA patient, especially if the SMA patient is in an advanced stage of the disease. Moreover, even in those muscles that are affected by SMA, not all neurons are dysfunctional (Fletcher et al., 2017. Nat Neuroscience 20 (7): 905-16; Mentis et al., 2011. Neuron 69 (3): 453-67) suggesting that the muscle weakness is produced by the dysfunction of a percentage of MNs rather than by its death. Thus, MN dysfunction and their death in SMA patients are two independent processes.

The SMN gene has been mapped by linkage analysis to a complex region in chromosome 5q. In humans, this region contains an approximately 500 thousand base pairs (kb) inverted duplication resulting in two nearly identical copies of the SMN gene. SMA is caused by an inactivating mutation or deletion of the telomeric copy of the gene (SMN1) in both chromosomes, resulting in the loss of SMN1 gene function. However, patients retain the centromeric copy of the gene (SMN2), and the copy number of the SMN2 gene in SMA patients generally correlates inversely with the disease severity; i.e., patients with less severe SMA have more copies of SMN2. Nevertheless, SMN2 is unable to compensate completely for the loss of SMN1 function due to alternative splicing of exon 7 caused by a translationally silent C to T mutation in exon 7. As a result, the majority of transcripts produced from SMN2 lack exon 7 (47 SMN2), and encode a truncated SMN protein that has an impaired function and is rapidly degraded.

The SMN protein is thought to play a role in RNA processing and metabolism, having a well characterized function of mediating the assembly of a specific class of RNA-protein complexes termed snRNPs. SMN may have other functions in MNs, however its role in preventing the selective degeneration of MNs is not well established.

In most cases, SMA is diagnosed based on clinical symptoms and by the presence of at least one copy of the SMN1 gene test. However, in approximately 5% of cases SMA is caused by mutation in genes other than the inactivation of SMN1, some known and others not yet defined. In some cases, when the SMN1 gene test is not feasible or does not show any abnormality, other tests such as an electromyography (EMG) or muscle biopsy may be indicated.

Several mouse models of SMA have been developed. In particular, the SMN delta exon 7 (Δ7 SMN) model (Le et al., Hum. Mol. Genet., 2005, 14:845) carries both the SMN2 gene and several copies of the Δ7 SMN2 cDNA and recapitulates many of the phenotypic features of Type 1 SMA. The Δ7 SMN model can be used for both SMN2 expression studies as well as the evaluation of motor function and survival. The C/C-allele mouse model (Jackson Laboratory strain #008714, The Jackson Laboratory, Bar Harbor, ME) provides a less severe SMA disease model, with mice having reduced levels of both SMN2 full length (FL SMN2) mRNA and SMN protein. The C/C-allele mouse phenotype has the SMN2 gene and a hybrid mSMN1-SMN2 gene that undergoes alternative splicing, but does not have overt muscle weakness. The C/C-allele mouse model is used for SMN2 expression studies.

SMA severity ranges from respiratory failure in the neonatal period (type 1-2) to mild muscle weakness noticed in adulthood (type 4). Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the MN cells deteriorate, symptoms appear shortly afterward. The severe forms of the disease are fatal and all forms have no known cure. The course of SMA is directly related to the rate of MN cell deterioration and the resulting severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. The clinical spectrum of SMA disorders has been divided into the following five groups:
Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing.
Type 1 SMA (Infantile SMA or Werdnig-Hoffmann disease) presents symptoms between 0 and 6 months. This form of SMA is also very severe. Patients never achieve the ability to sit, and death usually occurs within the first 2 years without ventilatory support.
Type 2 SMA (Intermediate SMA) has an age of onset at 7-18 months. Patients achieve the ability to sit unsupported, but never stand or walk unaided. Prognosis in this group is largely dependent on the degree of respiratory involvement.
Type 3 SMA (Juvenile SMA or Kugelberg-Welander disease) is generally diagnosed after 18 months. Type 3 SMA individuals are able to walk independently at some point during their disease course but often become wheelchair-bound during youth or adulthood.
Type 4 SMA (Adult onset SMA). Weakness usually begins in late adolescence in the tongue, hands, or feet, then progresses to other areas of the body. The course of adult SMA is much slower and has little or no impact on life expectancy.

SMA differs from other types of motor impairment, such as the impairments caused by spinal cord injury or stroke, in that non-functioning MNs are the root cause of the impairment due to SMA. The MNs of spinal cord injury or stroke patients have no underlying cellular pathophysiology. Thus, if the MNs of these patients receive an appropriate excitatory input, they are expected to produce a response. In contrast to the MNs of spinal cord injury or stroke patients, the MNs of SMA patients having cellular pathophysiology as a result of the genetic mutation of the SMN1 gene do not respond to excitatory input because the MNs themselves are dysfunctional or dead. Accordingly, there is no expectation that SCS, which aims to increase excitatory inputs to the spinal MNs, would produce an effect in an SMA patient similar to those effects observed in other conditions treated with SCS, such as spinal cord injury, stroke, and pain, where MNs are still functioning.

Traditional methods for treating motor impairment, such as exercise or physical therapy, alone may not be effective at treating SMA because neither exercise nor physical therapy can increase the firing rate of MNs to recruit muscle cells. The reduced firing rate of MNs in patients with SMA prevents the thorough engagement of muscle cells and causes a decrease in sensory inputs from the central nervous system. Thus, treatment methods to improve MN firing and function, and thereby recruit more muscle cells, are needed to improve the quality of life of patients with SMA. New genetic therapies have been shown to prevent the necessity of permanent breathing support and the death of neonatal patients (types 1-2). However, these genetic therapies have been unable to help types 3-4 patients with severe locomotion deficits in adulthood. Moreover, types 1-2 patients treated with genetic therapies also develop severe locomotion deficits. Additionally, other SMA therapies such as neuroprotective agents may not be fully effective in all patients. Thus, new therapies targeting motor impairments in SMA patients and improving the effectiveness of current therapies are needed to improve the quality of life of these patients.

### SUMMARY

Provided herein are implementations of a method for treating SMA in a subject. The method includes applying a therapeutically effective amount of an electrical stimulus to sensory neurons innervating a body region of the subject with a motor impairment due to SMA, wherein the electrical stimulus is applied with one or more electrodes controlled by a neurostimulator, and wherein application of the electrical stimulus treats the motor impairment due to SMA in the subject.

The foregoing and other objects, features, and advantages of the implementations will become more apparent from the following detailed description, which proceeds with reference to the accompanying figures.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1G illustrate various models of SCS potentiation of MN output, according to some embodiments.

FIGS. 2A-2C illustrate graphs describing force potentiation during simulated voluntary brain input, according to some embodiments.

FIG. 3A illustrates an SMA-affected neuron model with various ion channels, wherein the delayed rectifier potassium channels (K-dr) are blocked, according to some embodiments.

FIG. 3B illustrates a MN model with various ion channels, including a specific delayed rectifier potassium channel (Kv2.1), according to some embodiments.

FIGS. 4A-4C illustrate various HUMAC® Norm configurations to test the maximum torque in different joints in SMA patients receiving SCS stimulation of sensory neurons innervating lower limbs, according to some embodiments.

FIGS. 5A-5B illustrate an isokinetic machine (e.g., a HUMAC® Norm isokinetic machine) configured to test hip flexion and knee extension on an SMA patient, respectively, according to some embodiments.

FIG. 6 illustrates a comparison between the locations of the electrodes during the surgery and the locations after the surgery in an SMA patient, according to some embodiments.

FIGS. 7A-7C illustrate the various torque measurements over time for an SMA patient, according to some embodiments.

FIGS. 8A-8B illustrate that spinal cord stimulation increases maximum hip flexion during locomotion for an SMA patient, according to some embodiments.

FIGS. 9A-9B illustrate that spinal cord stimulation on the subject only temporarily disrupts balance for an SMA patient, according to some embodiments.

FIG. 10 illustrates that SCS robustly increases the maximum velocity of an SMA patient, according to some embodiments.

FIGS. 11A-11B illustrate a long-term effect of SCS in hip flexion for an SMA patient, according to some embodiments.

FIGS. 12A-12C illustrate long-term improvements in right knee extension for an SMA patient, according to some embodiments.

FIGS. 13A-13C illustrate long-term improvements in left knee extension for an SMA patient, according to some embodiments.

FIGS. 14A-14C illustrate long-term improvements in right hip flexion for an SMA patient, according to some embodiments.

FIGS. 15A-15C illustrate long-term improvements in left hip flexion for an SMA patient, according to some embodiments.

FIG. 16 illustrates manual muscle test scores for an SMA patient by muscle and session with and without stimulation, according to some embodiments.

FIG. 17 illustrates distances traveled by an SMA patient during a six-minute walk test across different sessions comparing stimulation off v. stimulation on, according to some embodiments.

FIG. 18A illustrates Hammersmith Functional Motor Scale Expanded scores for an SMA patient, according to some embodiments.

FIG. 18B illustrates Hammersmith Functional Motor Scale Expanded scores and Revised Hammersmith Scale scores for an SMA patient, according to some embodiments.

### DETAILED DESCRIPTION

### I. Introduction

Provided herein are methods for treating a subject with SMA. As discussed in detail herein, application of a therapeutically effective amount of an electrical stimulus to sensory neurons innervating a body region of the subject with a motor impairment due to SMA, via one or more electrodes controlled by a neurostimulator, treats the motor impairment due to SMA in the subject. Without being bound by theory, it is believed that application of the electrical stimulus to sensory neurons directly recruits mono- and poly-synaptic excitatory pathways in the spinal cord, which in turn increases the membrane potential and firing rate probability of spinal MNs innervating the body region of the subject with the motor impairment due to SMA. This recruitment of pathways may increase neuroplasticity, allowing the subject to regain motor functions. Further, in some implementations, application of the therapeutically effective amount of the electrical stimulus to the subject over time (for example, for at least 2 hours/day over a period of at least 6 months, or for at least 1 hour/day over a period of at least 1 month) may lead to ion channel remodeling on the MN membrane and a persistent increase in the firing rate probability of spinal MNs and improved motor impairment due, even in the absence of stimulation.

Thus, in an embodiment, therapeutic electrical stimuli may be administered to a subject by implanted or transcutaneous placement of electrodes under the control of an implanted or external neurostimulator. The electrodes and neurostimulator together comprise a system to provide SCS to a subject to improve motor impairment due to SMA such as, for example, muscle weakness, muscle control, and/or speech deficits. This system may be used in combination with other SMA therapies, such as targeted motor rehabilitation, to improve patient outcomes.

The application of SCS on SMA patients has produced results that are unexpected given the unique pathophysiology of SMA. As discussed above, SMA differs from other types of motor impairment, such as the impairments caused by spinal cord injury or stroke, in that non-functioning MNs are the root cause of the impairment due to SMA. Accordingly, there is no expectation that SCS, which aims to increase excitatory inputs to the spinal MNs, would produce an effect in an SMA patient similar to those effects observed in other conditions treated with SCS, such as spinal cord injury, stroke, and pain, where MNs are still functioning.

However, as discussed below, both significant immediate and long-term effects of SCS have been observed in an SMA patient treated according to the present disclosure. Unexpectedly, the magnitude of the immediate effects of SCS in SMA patients is at least as dramatic as those observed in patients with stroke and spinal cord injury. Even more remarkable is the magnitude of long-term effects observed after only 4 weeks of treatment according to the present disclosure, as discussed in detail below (e.g., measurements associated with MN excitability, MN firing rate, torques produced at different leg joints, EMG signals, maximum voluntary contraction during knee extension and/or hip flexion, hip flexion during locomotion, balance, sit-to-stand transitions, maximum running speed, range of motion, muscle strength (manual muscle test), gait (6-minute walk test), motor ability (Hammersmith Functional Motor Scale Expanded and Revised Hammersmith Scale tests), and leg circumferences). Specifically, improvements in muscle strength are of such a magnitude that they cannot be attributed to exercise only. For example, in one experiment, left hip flexion in an SMA subject more than doubled, even though the subject did not perform any hip strength training other than walking during the study. The subject's exercise level was unchanged from his exercise level before the study. These changes are also reflected in improvements observed in the clinical outcome tests such as the manual muscle test, the Revised Hammersmith Scale (RHS) test, and the 6-minute walk test. Moreover, the data does not indicate that a plateau has been reached, indicating that longer use of SCS may lead to even greater improvement in SMA patients.

Administration of SCS according to some embodiments of the present disclosure may artificially increase the activity on the same sensory afferent fibers (e.g., Ia) that are affected by SMA. This can have immediate (stimulation ON vs stimulation OFF during the study) and long-term effects on SMA patients (stimulation OFF pre study vs stimulation OFF post study). Without being bound by any particular theory, SCS may immediately increase the excitatory inputs to MNs and thus their firing rates. This may produce an immediate improvement in motor deficits. In the long-term, the artificial increase in sensory afferent activity may potentiate the affected sensory synapses and it may revert the maladaptive changes in MNs ion-channels thereby improving MN dysfunction that will result in measurable changes of motor function. Thus, SCS may address motor deficits produced by the decreases in the presynaptic activity of sensory afferent fibers. In some embodiments, SCS may be combined with pharmaceutical interventions designed to stop the disease progression.

### II. Acronyms

CST corticospinal tract
DRG dorsal root ganglion
EMG electromyography
MN motoneuron or motor neuron
SCS spinal cord stimulation
SMA spinal muscular atrophy
SMN1 survival motoneuron 1

### III. Summary of Terms

Unless otherwise noted, technical terms are used according to conventional usage. As used herein, the term “comprises” means “includes.” Although many methods and materials similar or equivalent to those described herein can be used, particular suitable methods and materials are described below. To facilitate review of the various implementations, the following explanations of terms are provided:

About: As used herein, the term “about” refers to an approximation of a qualitative or quantitative measurement. Whether the measurement is qualitative or quantitative should be clear from its context. With regard to quantitative measurements, “about” refers to plus or minus 5% of a reference value. For example, “about” 100 mA refers to 95 mA to 105 mA.

Dorsal rootlets: A small branch of a root of a sensory neuron that emerges from the posterior spinal cord and travels to the dorsal root ganglion.

Dorsolateral spinal cord: A region on the exterior surface of the spinal cord located between the dorsal midline and the point of entry of the dorsal rootlets into the main cord.

Electrical stimulus: The passing of various types of current selectively through one or more electrodes to a target location in a subject (for example, specific areas of the dorsolateral spinal cord).

Electrode: An electric conductor through which an electric current can pass. An electrode can also be a collector and/or emitter of an electric current. In some implementations, an electrode is a solid and comprises a conducting metal as the conductive layer. Non-limiting examples of conducting metals include noble or refractory metals and alloys, such as stainless steel, tungsten, platinum, iridium, tantalum, titanium, titanium nitride, and niobium. The electrodes can be either interconnected or independently wired.

Implanting: Completely or partially placing an electrode(s) or device containing the electrode(s) within a subject, for example, using surgical techniques. A device is partially implanted when some of the device reaches, or extends to the outside of, a subject. Implantable electrodes and devices may be implanted epidurally at the spinal cord, such as at the dorsolateral aspect of the spinal cord. An electrode or device can be implanted for varying durations, such as for a short-term duration (e.g., one or two days or less) or for long-term or chronic duration (e.g., one month, six months, one year, or more), as in a daily assistive device.

Motor impairment: The partial or total loss of function of a body part, for example, legs, feet, arms, hands, fingers, neck, and trunk (e.g., respiratory muscles). Particular motor impairments include loss of muscle strength, partial paralysis (paresis), loss of dexterity (such as hand finger movement), and uncontrollable muscle tone. A subject can exhibit multiple motor impairments as co-morbidities of SMA.

Motor threshold: The minimum thalamic stimulation intensity that can produce a motor output of a given amplitude from a muscle at rest (RMT) or during a muscle contraction (AMT).

Neurostimulator: A current or voltage-controlled electrical stimulation device. A neurostimulator controls the delivery of an electrical pulse, or pattern of electrical pulses, having defined parameters, for example and without limitation, pulse frequency, duration, amplitude, phase symmetry, duty cycle, pulse current, pulse width, and on-time and off-time. The controlled electrical pulse is delivered through one or more electrodes (for example, leadless electrode(s), or electrode(s) located at the end of a lead, a thin insulated wire) configured to apply the electrical stimulus to target tissue of a subject. A neurostimulator may comprise at least one multiple contact lead. Neurostimulators may be utilized to apply a series of electrical pulse stimuli (e.g., charge balanced pulses) through at least one electrode; for example and without limitation, low-frequency pulse train patterns, frequency-sequenced pulse burst train patterns (e.g., wherein different sequences of modulated electrical stimuli are generated at different burst frequencies), and phasic train patterns (e.g., wherein the stimulus control parameters change over the course of feedback from a subject's movement).

Perceptual threshold: The minimum electrical stimulation intensity necessary for a conscious human to be aware of a particular sensation caused by the electrical stimulation.

Sensory neurons: Also known as afferent neurons, sensory neurons are nerve cells within the peripheral nervous system responsible for converting stimuli from the environment of the neuron into internal electrical impulses and transmitting the impulse to the central nervous system.

Spinal Muscular Atrophy (SMA): A disease typically caused by an inactivating mutation or deletion in the SMN1 gene on both chromosomes, resulting in a loss of SMN1 gene function.

Subject: Living multi-cellular vertebrate organisms, a category that includes human and non-human mammals, including non-human primates, rats, mice, guinea pigs, cats, dogs, cows, horses, and the like. Thus, the term “subject” includes both human and veterinary subjects.

Therapeutically effective amount: An amount of a compound or treatment (or both) sufficient to provide a beneficial, or therapeutic, effect to a subject or a given percentage of subjects. Therapeutically effective amounts of a particular compound or treatment can be determined in many different ways, such as assaying for a reduction in a disease or condition (such as motor impairment due to SMA). Therapeutic compounds and treatments can be administered in a single application, or in several applications (e.g., chronically over an appropriate period of time). However, the effective amount can be dependent on the source applied, the subject being treated, the severity and type of the condition being treated, and the manner of administration.

Transcutaneous placement: Placing an electrode(s) or device containing the electrode(s) on or near the skin surface of a subject, for example, using non-invasive techniques. Electrodes may be applied to the skin surface of the subject to apply electrical stimulation, for example, under the control of an external neurostimulator. An electrode or device can be placed transcutaneously for varying durations, such as for a short-term duration (e.g., one or two days or less) or for long-term or chronic duration (e.g., one month, six months, one year, or more), as in a daily assistive device.

Treating/Treatment: With respect to a disease or condition (e.g., SMA), either term includes one or more of (1) preventing the disease or condition, e.g., causing the clinical symptoms of the disease or condition not to develop in a subject that may be exposed to or predisposed to the disease or condition but does not yet experience or display symptoms of the disease or condition, (2) inhibiting the disease or condition, e.g., arresting the development of the disease or condition or its clinical symptoms, and (3) relieving the disease or condition, e.g., causing regression of the disease or condition or its clinical symptoms.

More particularly, the treating or treatment of SMA denotes at least one or more of the following beneficial effects: a reduction in the loss of muscle strength, an increase in muscle strength, a reduction in muscle atrophy, a reduction in the loss of motor function, a reduction in contractures, an increase in MNs, a reduction in the loss of MNs, protection of SMN deficient MNs from degeneration, an increase in motor function, an increase in pulmonary function, a reduction in the loss of pulmonary function, and/or an increase in quality of life.

In further detail, treating or treatment of SMA refers to the functional ability or retention of the functional ability for a human infant or a human toddler to perform certain movements such as to sit up unaided, or for a human infant, a human toddler, a human child or a human adult to perform certain movements such as to stand up unaided, to walk unaided, to run unaided, to breathe unaided, to turn during sleep unaided, or to swallow unaided.

### IV. Stimulation of Sensory Neurons to Treat SMA

Provided herein are methods for treating a subject (for example, a human subject) with SMA. The methods may be utilized to treat (i.e., prevent, ameliorate, suppress, and/or alleviate) a motor impairment due to SMA in the subject. The method includes application of a therapeutically effective amount of an electrical stimulus to sensory neurons innervating a body region of the subject with a motor impairment due to SMA. The electrical stimulus may be applied, for example, with one or more electrodes controlled by a neurostimulator.

In an embodiment, applying the electrical stimulus to the sensory neurons increases the firing rate probability of spinal MNs innervating the body region of the subject with the motor impairment due to SMA. Without being bound by theory, it is believed that application of the electrical stimulus to sensory neurons may directly recruit mono- and poly-synaptic excitatory pathways in the spinal cord, which may indirectly increase the membrane potential and firing rate probability of spinal MNs innervating the body region of the subject with the motor impairment due to SMA. This recruitment of pathways may increase neuroplasticity, allowing the subject to regain motor functions.

Any appropriate subject with, or at risk of, a motor impairment due to SMA can be treated with the methods provided herein. The motor impairment can be in a limb of the upper or lower body, such as above or below the elbow, or above or below the knee, or including the entire arm or leg. The subject can have any of SMA types 1-4, such as type 1, type 2, type 3, or type 4. In some implementations, a subject with SMA is selected for treatment. The method can be in initiated at any time post-onset of the motor impairment in the subject, or even in advance of detectable motor impairment in an SMA patient at risk of motor impairment.

Application of the therapeutically effective amount of the electrical stimulus to the subject with SMA treats at least one motor impairment due to SMA in the subject. For example, application of the therapeutically effective amount of the electrical stimulus can result in a reduction in the loss of muscle strength, an increase in muscle strength, a reduction in muscle atrophy, a reduction in the loss of motor function, an increase in motor function, an increase in pulmonary function, and/or a reduction in the loss of pulmonary function.

In some embodiments, the motor impairment includes reduced control of a limb (such as an arm or leg) and the method provided herein increases control of the limb of the subject by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

In some embodiments, the motor impairment includes reduced postural balance and stability and the method provided herein increases postural balance and stability of the subject by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

In some embodiments, the motor impairment includes reduced leg torque and the method provided herein increases leg torque of the subject by at least 20% (such as at least 30%, at least 40%, at least 50%, at least 60%, at least 70%, at least 80%, at least 90%) relative to before the treatment as measured using a HUMAC® Norm system.

The one or more electrodes may be placed in any suitable position for applying the electrical stimulus to sensory neurons in the subject. In some implementations, the one or more electrodes are positioned to deliver an electrical stimulus to one or more sensory neurons innervating the body region with the motor impairment of the subject.

The spinal cord stimulation (SCS) system may comprise a neurostimulator and one or more spinal leads comprising a plurality of electrodes or contacts. Contacts of the SCS system can target specific muscles. For example, a contact located near the spinal segment L2 may target the hip flexor muscles, whereas a contact located near the spinal segment S1 may target the gluteus muscle and the ankle extensors. Accordingly, the location of the contacts or electrodes can be chosen to selectively target specific nerves innervating the muscles affected in SMA. For example, if a human subject has significant deficits in knee extensors and hip flexors, the stimulation system can be located to selectively target these muscles.

In some embodiments, the location for administering SCS can be optimized and fine-tuned by stimulating various contact points in an SMA patient, measuring electrical activity, such as electromyographic (EMG) signals, produced by the muscles (e.g., agonist and antagonist muscles of each joint), and evaluating the measurements. In one example, during surgery, the positions of the electrodes are determined by stimulating each contact point and recording EMG signals of muscles ranging from the trunk down to the ankles. Stimulation of a specific contact may begin at a low frequency (e.g., about 1 Hz) and progressively increase the amplitude of the stimulation until electrical activity associated with muscle activity in response to the stimulation is recorded. Based on the recorded electrical activity, the specific muscle(s) targeted by the specific contact can be identified, and thus, the stimulated contact points can be identified as suitable locations for the electrodes. The peak-to-peak amplitude of the EMG waveforms produced due to stimulation may be used to identify responses in the target muscles and thereby determine the correct locations of the muscles. For example, rostral contacts may produce their first waveforms in the hip flexors, whereas caudal contacts may produce their first waveforms in the calf muscles. Stimulations may be repeated until a location is identified where the electrodes activate, for example, from the hip muscles with the most rostral contacts to the calf muscles with the most caudal contacts. In other words, once the multiple contacts of the SCS system provide full coverage of the leg muscles (e.g., from hip to ankle) for the purpose of administering SCS, the position of the lead can be fixed. In some embodiments, the identified location can be used in a second SMA patient exhibiting similar symptoms without performing the above test on the second SMA patient. In some embodiments, the above test is performed during the surgery for implanting the electrodes.

In some implementations, the body region of the subject with the motor impairment is selected from the lower back, hip, leg, ankle, and foot. In such implementations, the one or more electrodes are positioned to apply an electrical stimulus to sensory neurons such as those of the T11-S1 nerve roots. For example, the one or more electrodes may be implanted at the sensory nerve or DRG, or implanted epidurally at dorsal rootlets or at the dorsolateral aspect of the spinal cord for sensory neurons of one or more of the T11-S1 nerve roots.

In some implementations, the body region of the subject with the motor impairment is selected from the upper arm, shoulder, arm, hand, and respiratory muscles (such as intercostal muscles or diaphragm). In such implementations, the one or more electrodes are positioned to apply an electrical stimulus to sensory neurons such as those of the C3-T2 nerve roots. For example, the one or more electrodes may be implanted at the sensory nerve or DRG, or implanted epidurally at dorsal rootlets or at the dorsolateral aspect of the spinal cord for sensory neurons of one or more of the C3-T2 nerve roots.

In some implementations, the body region of the subject with the motor impairment is selected from the chest, chest wall, abdomen, upper back, and middle back. In such implementations, the one or more electrodes are positioned to apply an electrical stimulus to sensory neurons such as those of the T3-T10 nerve roots. For example, the one or more electrodes may be implanted at the sensory nerve or DRG, or are implanted epidurally at dorsal rootlets or at the dorsolateral aspect of the spinal cord, for sensory neurons of the T3-T10 nerve roots.

In some embodiments, the placement of the electrodes during surgery can be adjusted to account for movement of the electrodes post-surgery. Such post-surgery movement may be variable and difficult to avoid with non-permanent implants. For example, the electrodes may move caudally and shift medially. Accordingly, based on predicted post-surgery movement of the electrodes, the electrodes can be implanted at adjusted locations during surgery such that, after the surgery, the electrodes would move to the predefined optimal locations. Additional details on the equipment and surgical procedures that can be used to acquire chronic electromyographic (EMG) recordings from leg muscles and to implant targeted spinal cord stimulation systems can be found in Capogrosso et al., 2018. Nature Protocols 13. 2031-2061, which is incorporated by reference herein.

Any suitable stimulation pattern may be used to treat the motor impairment in the subject. In some implementations, the electrical stimulus includes electrical pulses defined by parameters including, for example and without limitation, amplitude, pulse width, and pulse frequency. Such electrical pulses may include charge-balanced pulses, such as cathodic-first biphasic or monophasic charge balanced pulses. In these and further implementations, the electrical stimulus may be a continuous electrical stimulus, or a periodic stimulus.

The stimulation parameters of the SCS can be configured according to the techniques and values/ranges described herein. In one example, the electrical stimulus can be configured to comprise electrical pulses having an amplitude of about 10 μA to about 10 mA, a width between about 40 μs and about 2 ms, and/or a frequency of about 10 Hz to about 2000 Hz. In one example, the electrical stimulus can be configured to have a preferred frequency of about 40 Hz.

In particular examples, the electrical stimulus includes electrical pulses with an amplitude of about 10 μA to about 50 mA, such as about 10 μA to 10 mA, about 10 μA to about 1 mA, about 10 μA to about 100 μA, or about 100 μA to about 1 mA.

In particular examples, the electrical stimulus includes electrical pulses with pulse widths between about 40 μs and about 2 ms; for example, between 40 μs and 2 ms, between 100 μs and 2 ms, between 200 μs and 2 ms, between 300 μs and 2 ms, between 400 μs and 2 ms, between 500 μs and 2 ms, between 600 μs and 2 ms, between 700 μs and 2 ms, between 800 μs and 2 ms, between 800 μs and 2 ms, between 900 μs and 2 ms, between 1 ms and 2 ms, between 1.5 ms and 2 ms, between 80 μs and 1.5 ms, between 100 μs and 1.5 ms, between 200 μs and 1.5 ms, between 300 μs and 1.5 ms, between 400 μs and 1.5 ms, between 500 μs and 1.5 ms, between 600 μs and 1.5 ms, between 700 μs and 1.5 ms, between 800 μs and 1.5 ms, between 800 μs and 1.5 ms, between 900 μs and 1.5 ms, between 1 ms and 1.5 ms, between 1.5 ms and 2 ms, between 80 μs and 1 ms, between 100 μs and 1 ms, between 200 μs and 1 ms, between 300 μs and 1 ms, between 400 μs and 1 ms, between 500 μs and 1 ms, between 600 μs and 1 ms, between 700 μs and 1 ms, between 800 μs and 1 ms, between 800 μs and 1 ms, and between 900 μs and 1 ms.

In particular examples, the electrical stimulus includes a pulse frequency between about 10 Hz and about 2000 Hz; for example, between 20 Hz and 100 Hz, between 20 Hz and 90 Hz, between 20 Hz and 80 Hz, between 20 Hz and 70 Hz, between 20 Hz and 60 Hz, between 20 Hz and 50 Hz, between 20 Hz and 40 Hz, and between 20 Hz and 30 Hz.

In some implementations, the electrical stimulus includes electrical pulses having an amplitude of 10 μA to about 50 mA, a pulse width of between about 40 μs and about 2 ms, and a pulse frequency between about 10 Hz and about 2000 Hz. In some implementations, the electrical stimulus includes electrical pulses having an amplitude of 10 μA to about 10 mA, a pulse width of between about 40 μs and about 2 ms, and a pulse frequency between about 10 Hz and about 1000 Hz. In some implementations, the electrical stimulus includes electrical pulses having an amplitude of 100 μA to about 10 mA, a pulse width of between about 40 μs and about 500 μs, and a pulse frequency between about 10 Hz and about 1000 Hz.

In some implementations, bipolar and/or tripolar stimulation may be used. In some implementations, e.g., to target the right leg muscles, the spinal cord is stimulated using bipolar stimulation of the two most rostral contacts of the right lead. In some examples, the electrical stimulus includes electrical pulses having an amplitude of about 2 mA, a pulse width of about 400 μs, and a pulse frequency of about 40 Hz.

In some implementations, e.g., to target the left leg muscles, tripolar stimulation is used. In some examples, the electrical stimulus includes electrical pulses having an amplitude of about 3.7 mA, a pulse width of about 400 μs, and a pulse frequency of about 40 Hz.

In some implementations, the electrical stimulus includes electrical pulses having an amplitude of less than about 10 mA, a pulse width of between about 80 μs and about 2 ms, and a pulse frequency between about 20 Hz and about 100 Hz. In some examples, the electrical stimulus includes electrical pulses having an amplitude of between 0.5 mA and 5 mA, pulse widths between 80 μs and 200 μs, and a pulse frequency between 20 Hz and 80 Hz. For example, the electrical stimulus may include electrical pulses having an amplitude of between about 1.5 and about 3.5 mA, pulse widths between about 100 μs and about 200 μs, and a pulse frequency between about 40 Hz and about 80 Hz.

The electrical stimulus may be applied to the subject for any suitable amount of time needed to achieve a positive functional benefit for the patient. In some implementations, the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz. In some implementations, the electrical stimulus is applied for at least 1 hour/day over a period of at least 1 month. In some implementations, the electrical stimulus is applied for at least 2 hours/day over a period of at least 6 months.

In particular implementations, the stimulation is applied at or below the subject's motor threshold and/or at or below the subject's perceptual threshold. For example, the stimulation may be applied below the motor threshold and below the perceptual threshold; below the motor threshold, but at or above the perceptual threshold; or below the perceptual threshold, but at or above the motor threshold.

The SCS system can be configured within a few days to provide electrical spinal cord stimulation protocols that allow control over the degree of extension and flexion of muscles during motion of that limb, e.g., each leg during locomotion and real-time processing of gait kinematics and locomotor performance. In some implementations, the subject may be monitored and tested to establish parameters for the electrical stimulation based on the subject's motor disorder and motor impairment; for example, by monitoring one or more motor output(s) that provide a measurement of the extent of the motor impairment and the subject's response to stimulation. In some implementations, electrical stimulation by the electrode(s) is delivered to sensory neurons in the subject while the subject performs a voluntary activity or task affected by the subject's motor impairment; for example, forelimb tasks (e.g., reaching, grabbing, picking with opposable thumbs, grip squeezing, and fine motor tasks involving precision finger movements) or lower limb tasks (e.g., walking, jumping, leg extensions).

Once configured, stimulation bursts are delivered over specific spinal cord locations with precise timing that reproduces the natural spatiotemporal activation of MNs during locomotion. These protocols can also be easily adapted for the safe implantation of systems in the vicinity of the spinal cord and to provide SCS involving real-time movement feedback and closed-loop controllers, as discussed below. In some implementations, the parameters of the electrical stimulus controlled by the neurostimulator are adjusted according to changes in the one or more motor output(s) that are monitored while the subject performs a specific task, for example, so as to improve the motor outputs, thereby treating the subject's motor impairment. In particular implementations, the adjusted neurostimulator is part of a daily assistive device to treat the subject over an extended period of time. In specific implementations, the operation of the device and/or the neurostimulator can be at least partially under the control of the subject once the subject is released from a clinical setting. In these and further implementations, the subject is taught how to use the device and/or the neurostimulator. In some non-limiting implementations, this treatment may increase inputs on the membrane of the spinal MNs by means of direct recruitment of sensory afferents from the electrical pulses and or may lead to ion channel remodeling on the MN membrane to increasing firing rate probability of spinal MNs and/or improving motor impairment due to SMA, including when the electrical stimulus is no longer applied to the patient. Accordingly, the disclosure includes methods of increasing the firing rate of motoneurons impaired by SMA.

Stimulation of sensory afferents using implanted electrodes is an advanced neurosurgical procedure involving the implantation of one or more electrode(s) that deliver an electrical stimulus under the control of an externalized or implanted neurostimulator unit. Implantation of the electrode(s), and/or a neurostimulator in examples where the neurostimulator is not externalized, is typically performed by a clinical team including neurologists, neurosurgeons, neurophysiologists, and other specialists trained in the assessment, treatment, and care of neurological conditions. Typically, following selection of an appropriate subject and determination of the target area of the subject to be stimulated, precise placement of at least one electrode in the area of the patient's sensory afferents (such as the dorsolateral aspect of the spinal cord) is carried out in an operating room setting, typically utilizing spinal cord imaging technology. After administration of local anesthesia, the subject undergoing electrode implantation experiences little discomfort, and may be kept awake during the implantation procedure to allow communication with the surgical team.

Some implementations herein employ an implant that includes one or more electrodes and/or neurostimulator implanted (e.g., fully or partially implanted) in the subject. Further implementations herein employ an implant that includes one or more magnets or optical fibers, and/or a neurostimulator implanted in the subject.

Numerous types and styles of implants (for example, implants including one or more electrodes for providing an electrical stimulus) are available and known to those in the art. Any implant for specific stimulation of sensory neurons in a subject may be utilized in specific implementations. In some implementations, more than one electrode is implanted, such as an array of electrodes. In additional implementations, a device is provided that can include one or more electrodes. Non-limiting examples include, electrode arrays, penetrating microarrays (e.g., Utah and Michigan microarrays), microwire electrodes and arrays, nerve cuffs, and paddle arrays.

In some implementations, the one or more electrodes are implanted at the dorsal rootlets of the one or more sensory neurons innervating the body region with the motor impairment of the subject. In some implementations, the one or more electrodes are implanted at the dorsolateral aspect of the spinal cord adjacent to the dorsal roots of the one or more sensory neurons innervating the body region with the motor impairment of the subject. In some implementations, the one or more electrodes are implanted at the dorsal root ganglia of the one or more sensory neurons innervating the body region with the motor impairment of the subject. In some implementations, the one or more electrodes are contained in a cuff that surrounds or at least partially surrounds a peripheral nerve containing the sensory neurons innervating the body region of the subject with the motor impairment due to SMA. In further implementations, the one or more electrodes penetrate a peripheral nerve or dorsal root ganglion containing the sensory neurons innervating the body region of the subject with the motor impairment due to SMA.

In some implementations, epidural electrical stimulation (EES) targeting the dorsal rootlets using a paddle electrode array is utilized in the disclosed methods, for example, as described in Rowald et al., 2022, Nat. Medicine, 28: 260-271 and Wagner et al., 2018, Nature, 563: 65-71, each of which is incorporated by reference herein. Additional non-limiting examples of paddle arrays and their use are provided, for example, in US2009/0351221, US2019/0366077, each of which is incorporated by reference herein.

In some implementations, circuitry is implanted connecting a neurostimulator to the one or more electrodes. In particular implementations, the circuits are fully implanted (typically in a subcutaneous pocket within a subject's body), or are partially implanted in the subject. The operable linkage of the neurostimulator to the electrode(s) can be by way of one or more leads, although any operable linkage capable of transmitting a stimulation signal from the circuitry to the electrodes may be used in specific implementations.

Any suitable control system can be used with the electrodes to apply the electrical stimulus to the subject. Non-limiting examples of controllable neurostimulation systems are provided in US2020/0254260, US2020/0360693, US2020/0360697, US2020/0152078, U.S. Pat. Nos. 10,252,065, 10,799,702, US2021/0016093, and US2020/0391030, each of which is incorporated by reference herein. Further, non-limiting examples of closed-loop neurostimulation systems are provided in U.S. Pat. Nos. 10,265,525, 10,279,167, 10,279,177, 10,391,309, 10,751,539, 10,981,004, and US2020/0147382, each of which are incorporated by reference herein.

Post-operative control of selective electrical stimulation by the implanted electrode is provided in some implementations by a neurostimulator that may be externalized or implanted; for example, subcutaneously (e.g., in the chest or belly of the subject). In some implementations, disclosed methods are affected by the use of an implanted neurostimulator that controls the stimulation (e.g., electrical stimulation via one or more implanted electrode(s)) according to predetermined parameters or parameters determined by feedback in a closed-loop system. In particular implementations, the one or more electrodes and the neurostimulator comprise a daily assistive device that improves muscle weakness in an affected limb of the subject.

Following recovery from the implantation, surgery, and connection of electrode leads to the neurostimulator, the subject may be monitored and tested to establish parameters for the electrical stimulation based on the subject's motor disorder and motor impairment; for example, by monitoring one or more motor output(s) that provide a measurement of the extent of the motor impairment and the subject's response to stimulation. In some implementations, electrical stimulation by the implanted electrode(s) is delivered to sensory neurons in the subject while the subject performs a voluntary activity or task affected by the subject's motor impairment; for example, forelimb tasks (e.g., reaching, grabbing, picking with opposable thumbs, grip squeezing, and fine motor tasks involving precision finger movements) or lower limb tasks (e.g., walking, jumping, leg extensions).

In some implementations, a transcutaneous electrical stimulation system is used to apply the electrical stimulus to the sensory neurons in the subject. A non-limiting example of a transcutaneous stimulation system is provided in U.S. Pat. No. 10,806,927, which is incorporated by reference herein. In such transcutaneous implementations, the electrical stimulus may comprise electrical pulses having an amplitude of about 10 μA to about 100 mA, a width between about 40 μs and about 2 ms, and a frequency of about 10 Hz to about 10000 Hz.

In particular implementations, methods disclosed herein can be used in combination with protocolled physical rehabilitation exercises to improve long-term outcomes.

### EXAMPLES

The following examples are provided to illustrate particular features of certain implementations, but the scope of the claims should not be limited to those features exemplified.

### Example 1

Biophysical model of spinal cord stimulation to treat SMA This example provides a biophysical model illustrating the effectiveness of sensory neuron stimulation therapy for SMA patients, as well as stimulation protocols for treating SMA patients (such as type 3 and type 4 SMA patients) with stimulation of the dorsolateral aspect of the spinal cord.

The application of SCS on SMA patients has produced results that are unexpected results given the unique pathophysiology of SMA. Although SMA patients generally have an intact corticospinal tract, unlike patients with spinal cord injury or stroke, many, if not all, MNs are non-functioning or dead in an SMA patient, especially if the SMA patient is in an advanced stage of the disease, making them functionally inert. Accordingly, there is no expectation that SCS, which aims to increase excitatory inputs to the spinal MNs, would produce any effect to an SMA patient because the MNs in the SMA patient are already non-functioning or dead. There is certainly no expectation that the application of SCS to treat SMA would produce the effects observed in other conditions treated with SCS, such as spinal cord injury, stroke, and pain, where MNs are still functioning.

Surprisingly, however, as discussed below, both significant immediate and long-term effects of SCS have unexpectedly been observed in SMA patients treated according to the present disclosure. Although experiments with SMA mouse models have shown a decrease in presynaptic activity from sensory afferent fibers, the present disclosure demonstrates that by artificially increasing the activity at the sensory afferent fibers through SCS, a therapeutically effective amount of SCS appropriately applied, such as at the dorsolateral aspect of the spinal cord, may increase the excitability of the MNs through the remaining excitatory connections and may generate long-term potentiation of the affected synapses that may contribute to reversing the electrical changes in SMA-affected MNs. Moreover, therapeutically effective amount of SCS may also potentiate the firing rate of SMA-unaffected MNs, producing a more robust effect.

### Network Model of the Spinal Cord Circuitry

### Healthy Neuron Model

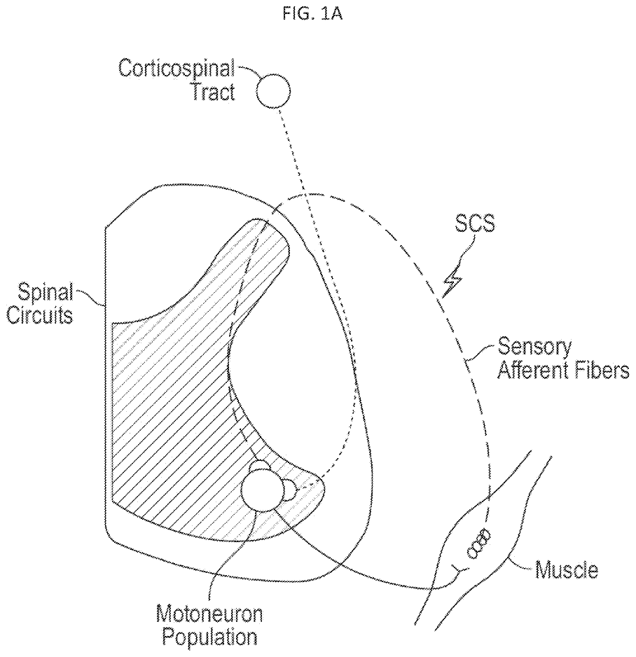

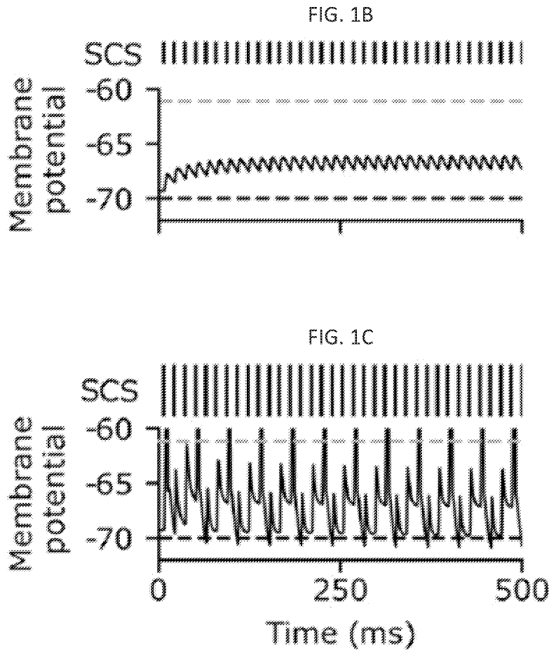

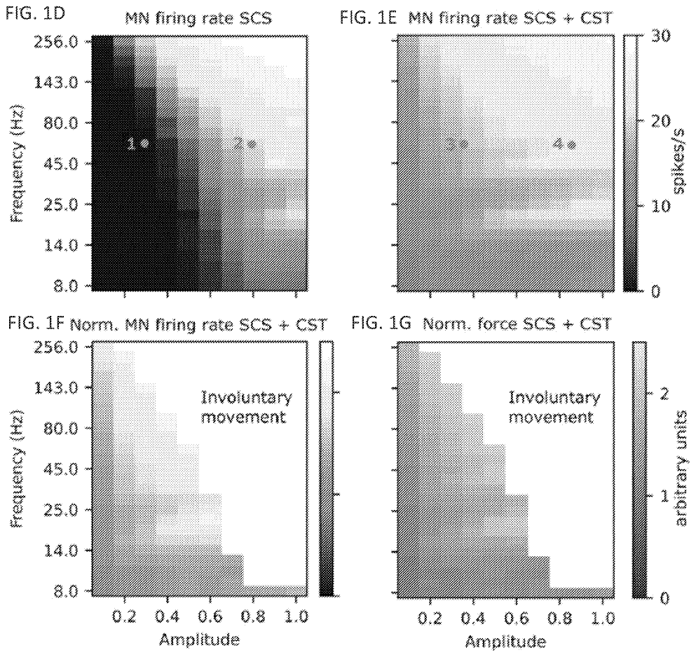

FIG. 1A illustrates a network model diagram, in which MNs receive excitatory inputs from the corticospinal tract and the spinal cord stimulation (SCS), such as dorsolateral SCS, through the recruitment of sensory afferent fibers. As shown in FIG. 1A, a biophysical model was built with a population of healthy MNs (N=169 modified Hodgkin-Huxley neurons, McIntyre et al., 2002. J Neurophysiology 88 (4):1592-1604). Each MN receives excitatory inputs from the corticospinal tract (CST, N=110) and the SCS through the recruitment of sensory afferent fibers (N=60) (see Capogrosso et al., 2013. J Neuroscience. 33(49):19326-40; Gerasimenko et al., 2006. J Neuroscience Methods 157 (2): 253-63; Hofstoetter et al., 2015. J Neurophysiology 114 (1): 400-410; Rattay et al., 2000. Spinal Cord 38 (8): 473-89). The difference between both excitatory sources is that the CST was modeled as a population of Poisson neurons while SCS had its own frequency and amplitude. To investigate the restoration of movement during SCS, the force produced by the MN pool was quantified in arbitrary units (Fuglevand et al., 1993. J Neurophysiology, 70(6):2470-2488). In contrast to the CST that represents voluntary inputs from the brain to the MNs, SCS is controlled by the experimenter. Thus, to improve voluntary movement, SCS should potentiate the MNs firing rate only when the CST is active.

### SMA Neuron Model

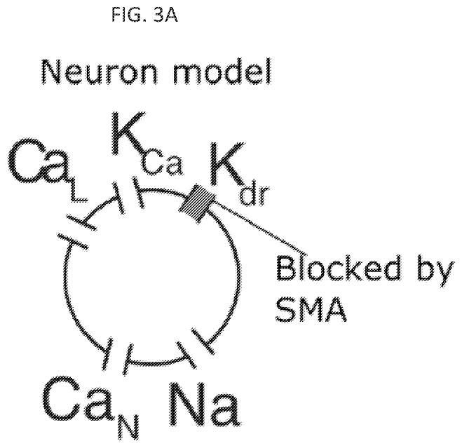

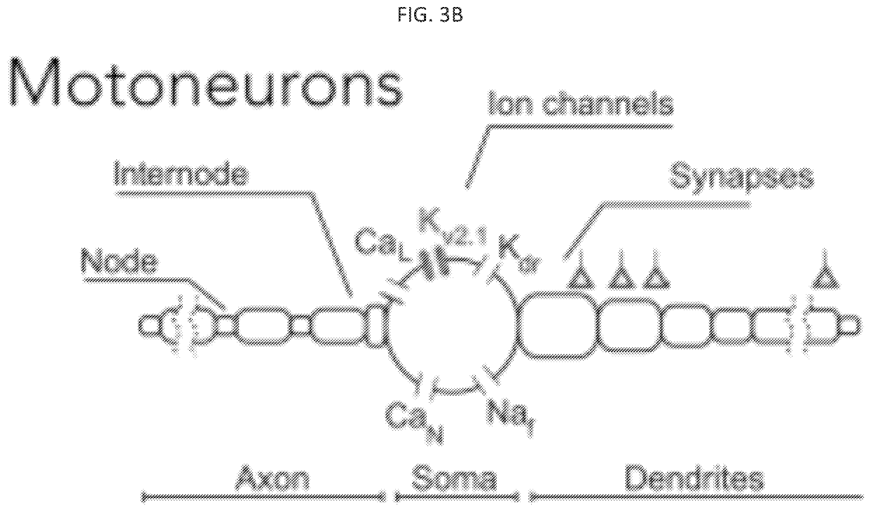

FIG. 3A illustrates an SMA-affected neuron model with various ion channels, wherein the delayed rectifier potassium channels (K-dr) are blocked. For the healthy neuron model, the established approach to describe the dynamics of the membrane potential as a function of the different ion channels is the Hodgkin-Huxley model. In this model, the membrane potential (V) is described by:

$$C \frac{dV}{dt} = {\sum }_{ions} {g}_{x} ( V - {V}_{x} ) + I$$

where C is the membrane capacity, I is current artificially injected to the neuron and g is the conductance x of the ion-channel x that, in general, depends on the membrane potential and ion concentrations. Following previous literature (Booth et al., 1997, J Neurophysiology 78 (6): 3371-85; McIntyre et al., 2002. J Neurophysiology 88 (4):1592-1604; Moraud et al., 2016. Neuron 89 (4):814-28), the following ion channels are included: delayed rectifier sodium (Na) and potassium (K-dr), N-like calcium (Ca-N), L-like calcium (Ca-L) and calcium-dependent potassium (K(Ca)), as shown in FIG. 3A. The model is implemented in a neuron stimulation environment designed for modeling individual neurons and networks of neurons referred to herein as NEURON (Hines and Carnevale. 1997. Neural Computation 9(6): 1179-1209).

FIG. 3B illustrates a MN model with various ion channels, including a specific delayed rectifier potassium channel (Kv2.1). Without being bound by any particular theory, the SMA-induced loss of sensory afferent inputs (e.g., Ia) may change the Kv2.1 channels of MNs into a dysfunctional state with increased refractory periods (e.g., slower firing rates) and/or increased input resistance. Additionally, deficits in MN function may be reverted by increasing sensory afferent inputs (e.g., via SCS) into SMA-affected MNs, which decreases the input resistance of the MNs. Furthermore, SCS may immediately increase sensory afferent inputs, thereby increasing the firing rates of SMA-affected MNs. Over time, SCS may rescue MN function by changing the Kv2.1 channels of MNs to their healthy, functional state.

Spinal Cord Stimulation Increases the Excitability of MN without Producing Involuntary Movement

FIGS. 1B-1G illustrate various models of SCS potentiation of MN output. In all panels of FIGS. 1D-1G, the “amplitude” unit is the percentage of sensory afferent fibers recruited by the SCS. FIG. 1B plots the membrane potential (mV) of a MN stimulated with a frequency of 69 Hz and an amplitude that recruited 30% of the sensory afferent fibers. Note that the parameters illustrated in FIG. 1B correspond to the point labeled “1” on FIG. 1D. The black dashed line shows the resting potential of the MN membrane potential, while the gray dashed line shows the MN spike threshold. For the appropriate parameters, SCS can increase the excitability of MNs without generating action potentials. FIG. 1C plots the membrane potential (mV) of a MN stimulated with a frequency of 69 Hz and an amplitude that recruited 80% of the sensory afferent fibers. Note that the parameters illustrated in FIG. 1C correspond to the point labeled “2” on FIG. 1D. FIG. 1D, which illustrates a gradient plot, plots MN firing rate as a function of SCS parameters without input from the corticospinal tract (CST), which shows that SCS by itself produces involuntary movements (MNs firing rate >8 Hz). Greater spikes per second is illustrated by the darker shading in each of FIGS. 1D-1F on a scale of 0-30 spikes/s. FIG. 1E, which illustrates a gradient plot, plots MN firing rate as a function of SCS parameters with input from the CST. FIG. 1F, which illustrates a gradient plot, plots MN firing rate for combinations of SCS parameters that generate only voluntary movement normalized by the MNs firing rate without SCS. FIG. 1G, which illustrates a gradient plot, plots how SCS potentiates the force generated by the MNs population. The “arbitrary units” scale of FIGS. 1F and 1G represent how many times larger the firing rate with SCS is than the firing rate without SCS. The upper end of the “arbitrary units” scale is 2.2 times. Thus, these figures show that MN firing rate is up to 2.2 times larger with SCS than without.

To study the effect of continuous SCS on the MNs firing rate, a range of stimulation parameters was explored. In general, the firing rate of MNs increased as a function of both SCS frequency and amplitude, as shown in FIG. 1D. However, a local maximum at 25 Hz was identified, which is related to time constants of the modified Hodgkin-Huxley neuron model (McIntyre et al., 2002. J Neurophysiology 88 (4):1592-1604). SCS was able to increase the firing rate of the MNs above the minimum firing rate to produce movement (>8 Hz) (Monster and Chan. 1977. J Neurophysiology 40 (6): 1432-43), as shown in FIGS. 1C and 1D. Any combination of SCS parameters that generated movement without the participation of the CST (i.e., involuntary movement) were discarded for the restoration of voluntary movement. However, SCS was also able to increase MN excitability (i.e., depolarize the membrane potential) without producing involuntary movement (i.e., MN firing rate <8 Hz), as shown in FIGS. 1B and 1D. This increase of the MN excitability represents a mechanism to potentiate CST inputs.

### SCS Potentiates Supraspinal Inputs Increasing MN Firing Rate

### Healthy Neuron Model

Having shown that SCS can increase MN excitability in the healthy model, whether SCS can also potentiate CST inputs to activate MNs and produce force was assessed. To perform this assessment, the input from the CST was fixed to generate a relatively low firing rate (9.3 Hz) in the MNs. The MNs firing rate was computed while systematically varying the SCS parameters (frequency and amplitude). For a range of these parameters, SCS potentiated the inputs from the CST to increase the firing rate of the MNs without generating involuntary movement, as shown in FIGS. 1E and 1F. The enhanced firing rate of the MN with SCS produced a force up to 2.2 times stronger than the firing rate generated by the same CST inputs without SCS, as shown in FIG. 1G.

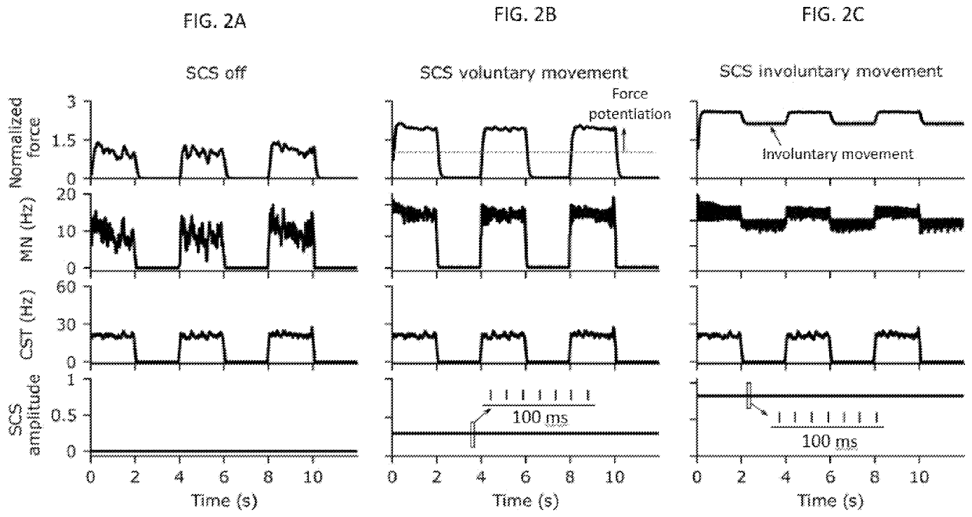

FIGS. 2A-2C illustrate graphs describing force potentiation during simulated voluntary brain input. FIG. 2A plots the following network activity parameters: the SCS amplitude, the firing rate of the corticospinal tract (CST) in Hz, MN firing rate in Hz, and the force normalized by the mean force applied. For the network activity of FIG. 2A, SCS stimulation is not applied, and a CST input of 22 Hz is applied. FIG. 2B plots network activity with SCS applied in the voluntary movement region. Note that the parameters illustrated in FIG. 2B correspond to the point labeled “3” on FIG. 1E. MNs firing rate and thus force are potentiated with respect to SCS off. FIG. 2C plots network activity with SCS applied in the involuntary movement region. Note that the parameters illustrated in FIG. 2C correspond to the point labeled “4” on FIG. 1E. MNs firing rate is high during both phases: with and without inputs from the CST.

To further understand the potentiation of CST inputs driven by SCS, an oscillatory force task was modeled where CST inputs arrived at the MNs in a periodic fashion, as shown in FIGS. 2A-2C. The differences between the voluntary and the involuntary movement regions were assessed. In the voluntary movement region, SCS increased the excitability of MNs producing firing rates higher than those observed without SCS. However, SCS did not increase the firing rate of the MNs during periods without CST inputs, as shown in FIGS. 2A and 2B. In the involuntary movement region, SCS increased the firing rate during the entire trial also during periods without inputs from the CST, as shown in FIG. 2C. Together these results show that, in the biophysical model of a healthy spinal cord, it is possible to potentiate the inputs from the CST to generate enhanced firing rate of the MNs, and thus a stronger force.

### SMA Neuron Model

A series of experiments with an SMA mouse model have shown that SMA affected MNs have dysfunctional electrical properties (Mentis et al., 2011. Neuron 69 (3): 453-67; Fletcher et al., 2017. Nat Neuroscience 20 (7): 905-16) due to a decrease in the synapses activity from the sensory afferent fibers and a block of delayed rectifier potassium channels (Fletcher et al., 2017. Nat Neuroscience 20 (7): 905-16; Simon et al., J Neuroscience. 41(2):376-389, 2021). The downregulation of the K-dr channel, as shown in FIG. 3A, decreases the excitability of the MNs. Thus, to model the SMA-affected MNs, the synaptic weights (i.e., the strength of the synapses) are decreased from the sensory afferent fiber and the conductance of the delayed rectifier potassium channel, as shown in FIG. 3A. This neuron model may be validated by reproducing the following three dysfunctional electrical properties of the SMA-affected MNs:
(1) High input resistance: Within the NEURON simulation environment, which is designed for modeling individual neurons and networks of neurons, it is easy to inject current directly into the MN and compute the input resistance as the slope of the voltage-current function. The block of the potassium channels will decrease the outflux of potassium ions, thereby increasing the input resistance.
(2) Reduced rheobase: As in (1), current is injected into the MN from which it is possible to compute the minimum input current to generate an action potential. Similarly, decreasing the outflux of potassium ions depolarizes the membrane potential faster, decreasing the injected current needed to trigger an action potential.
(3) Low firing rate induced by a current higher than the current needed to induce repeating firing: Injecting an artificial current above the threshold for repeating firing rate of the SMA-affected MN tests whether the block of the potassium channels is enough to reduce the firing rate. Potassium channels open after the action potential to repolarize the membrane potential. With their block the repolarization phase will be slower than in a healthy MN, decreasing the output firing rate (Fletcher et al., 2017. Nat Neuroscience 20 (7): 905-16).

### Example 2

### Spinal Cord Stimulation to Treat SMA

This example describes a particular method that can be used to treat SMA in a subject by applying a therapeutically effective amount of an electrical stimulus to the dorsolateral aspect of the spinal cord containing sensory neurons innervating a body region of the subject with motor impairment due to SMA. Although particular methods and protocols are provided, one skilled in the art will appreciate that variations can be made without substantially affecting the treatment.

A human patient with Type 3 or 4 SMA aged 22 that shows quantifiable motor deficits of the legs but is able to stand independently is selected for treatment. Percutaneous, bilateral, linear spinal leads are implanted in the patient near the lumbar spinal cord for a period of up to 29 days.

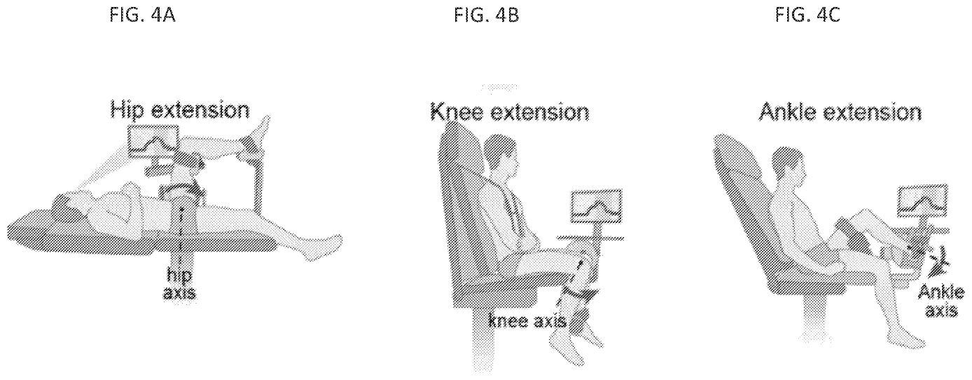

To quantify immediate motor improvement driven by the SCS, maximum torques produced at different joints such as the hip, knee and ankle during isometric movements are measured. The HUMAC® Norm system is used for these measurements. With this system, the patient can be placed in different positions to evaluate the maximum torque of such joints (FIGS. 4A-4C). FIG. 4A illustrates hip extension, FIG. 4B illustrates knee extension, and FIG. 4C illustrates ankle extension.

During the assessment, the patient produced a progressive contraction from rest to maximum strength, where he receive real-time torque visual feedback. The same assay is repeated to systematically explore different SCS parameters, including without SCS, to determine parameters that are most effective to reduce motor impairment in the patient. As an example, an electrical stimulus of electrical pulses having an amplitude of about 10 μA to about 50 mA, a width between about 40 μs and about 2 ms, and a frequency of about 10 Hz to about 2000 Hz can be applied. Additionally, the electrical activity produced by the agonist and antagonist muscles of each joint using surface electromyography (EMG) activity may be recorded. Applying appropriate SCS parameters to provide a therapeutically effective amount of electrical stimulation is expected to increase the maximum torque and the EMG activity.

This example aims to measure short-term and long-term effects of SCS on SMA. First, the example measures the immediate effects of SCS on SMA by turning on and off stimulation in the same session and measuring effects of SCS that are only present during stimulation and disappear when stimulation is turned off. Second, the example measures the long-term effects of SCS on SMA by comparing performance of a patient without stimulation over different sessions. Further, various clinical outcome tests are performed.

Both immediate and long-term effects of SCS on the SMA patient were observed. In contrast to what would be expected in SMA-affected patients, the magnitude of the immediate effects is similar to those observed in patients with stroke and spinal cord injury. Moreover, the magnitude of long-term effects observed over only 4 weeks of trial is unexpected for patients with non-functioning MNs, who experienced improvements in muscle strength beyond what a normal SMA-affected patient is expected to achieve. These improvements in muscle strength are too high to be attributed to exercise only. For example, left hip flexion of the patient more than doubled, but the patient did not perform any hip strength training other than walking during the study. The patient's exercise level was unchanged from his exercise level before the study. These changes were reflected in improvements in the clinical outcome tests such as the manual muscle test, the Hammersmith Functional Motor Scale Expanded (HFMSE), the Revised Hammersmith Scale (RHS), and the 6-minute walk test for the patient. Moreover, the data does not indicate that a plateau has been reached, indicating that longer use of SCS may lead to even greater improvement.

Over the course of 4 weeks, SCS therapy increases leg joint torques (up to +180%) and MN firing rates for the patient, indicating that SCS is improving MN function while reporting no side effects. These results indicate that SCS can potentially increase the quality of life of severe and mild SMA patients by improving muscle strength and gait. Such improvements of this magnitude within such a short time frame observed in patients with SMA are unexpected. The observed improvement in muscle strength of this magnitude, and in such a short time frame, may be indicative of a disease-modifying effect. Without being bound to any particular theory, the improved strength may be driven by rescued MN function in response to increase afferent inputs driven by SCS targeting the hip flexors and knee extensors afferents in the patient. Spinal reflexes and single MN firing rates obtained with HD-EMG recordings were analyzed to corroborate the conclusion.

### Initial Configuration and Optimization

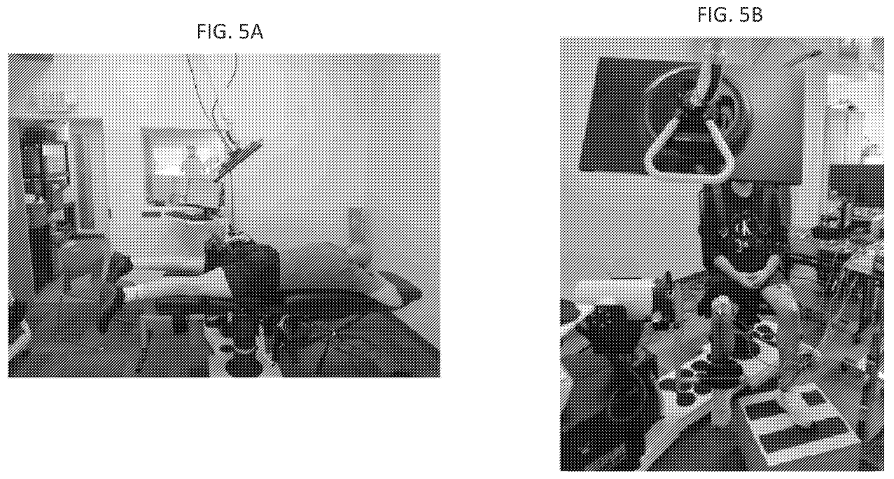

Before implantation of electrodes, the initial performance of a patient can be measured. The HUMAC® Norm system can be used for these measurements. With this system, the patient can be placed in different positions to evaluate the maximum torque of different joints. FIGS. 5A and 5B illustrate an isokinetic machine (e.g., a HUMAC® Norm isokinetic machine) configured to test hip flexion and knee extension on a human patient, respectively. In particular, the maximum torques produced by the patient during the extension and/or flexion at knee, hip, and ankle can be measured.

Bilateral linear SCS leads are implanted in the epidural space from T11 to L1 vertebrae. To target the right leg muscles, in one example, the spinal cord is stimulated using bipolar stimulation of the two most rostral contacts of the right lead. The SCS parameters in this example are 2 mA, 40 Hz, and 400 μs pulse width. To target the left leg muscles, in one example, tripolar stimulation is used. The SCS parameters in this example are 3.7 mA, 40 Hz, and 400 μs pulse width.

SCS can be administered to target muscles affected by SMA. In the example, it is found that the SMA-affected patient has significant deficits in knee extensors and hip flexors, consistently with his Type 3 diagnosis. Further, the patient has slightly more deficits in the left leg. Accordingly, the stimulator can be programmed to selectively target these muscles. In the example, all experiments are performed over the course of 4 weeks and a total of 19 sessions. The experiments are performed daily for 5 days per week until the date on which the electrodes are explanted. Each session lasts 4 hours with approximately 2 to 3 hours of time-on-task, with an estimated dose of stimulation active for 2 hours per day during these sessions.

The location for administering SCS can be optimized and fine-tuned by stimulating various contact points, measuring electrical activity produced by the muscles (e.g., agonist and antagonist muscles of each joint), and evaluating the measurements. Maximum torque and the EMG activity are then measured to determine a therapeutically effective amount of electrical stimulation using appropriate SCS parameters. In the example, during surgery, the positions of the electrodes are determined by stimulating each contact and recording EMG signals of muscles ranging from the trunk down to the ankles. Stimulations are repeated until a location is identified where the electrodes activate the hip muscles with the most rostral contacts and the calf muscles with the most caudal contacts.

These implants can be used to configure electrical spinal cord stimulation procedures that allow control over the degree of extension and flexion of each leg during locomotion. This protocol uses real-time processing of gait kinematics and locomotor performance, and can be configured within a few days. Once configured, stimulation bursts are delivered over specific spinal cord locations with precise timing that reproduces the natural spatiotemporal activation of MNs during locomotion. These protocols can also be easily adapted for the safe implantation of systems in the vicinity of the spinal cord and to conduct experiments involving real-time movement feedback and closed-loop controllers, as discussed below.

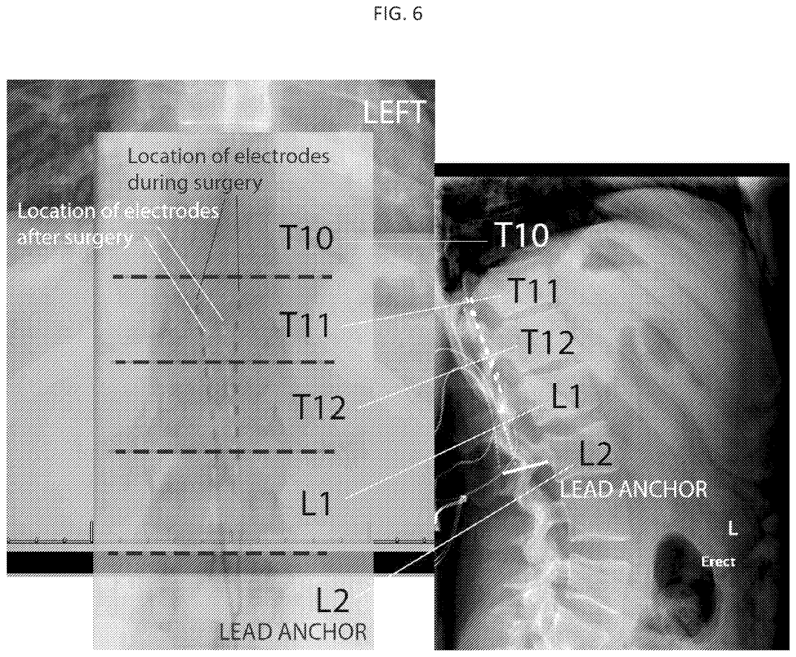

In some embodiments, the placement of the electrodes during surgery can be adjusted to account for movement of the electrodes post-surgery. Such post-surgery movement may be variable and difficult to avoid with non-permanent implants. For example, the electrodes may move caudally and shift medially. FIG. 6 illustrates a comparison between the locations of the electrodes during the surgery (in black) and the locations after the surgery (in white). In this particular example, the change of locations does not prevent the stimulation to the correct group of muscles. In some embodiments, based on predicted post-surgery movement of the electrodes, the electrodes can be implanted at adjusted locations during surgery such that, after the surgery, the electrodes would move to the predefined optimal locations. Additional details on the equipment and surgical procedures that can be used to acquire chronic EMG recordings from leg muscles and to implant targeted spinal cord stimulation systems can be found in Capogrosso et al., 2018. Nature Protocols 13. 2031-2061, which is incorporated by reference herein.

### Observed Immediate Effects

In the example, the immediate effects of SCS are measured in terms of maximum voluntary contraction, hip flexion during locomotion, balance, sit-to-stand transitions, and maximum speed. Each measurement is discussed below. It should be appreciated that different metrics can be used depending on the targeted muscle(s) and conditions of a patient.

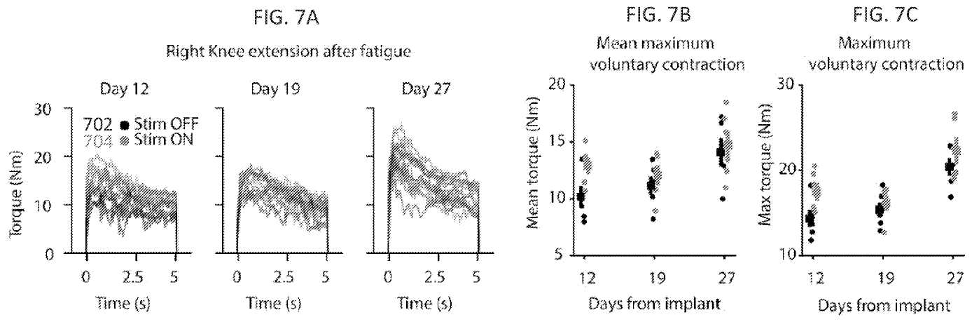

Maximum Voluntary Contraction: To evaluate maximum voluntary contraction, the patient is asked to produce his maximum voluntary isometric contraction (MVC) during knee extension for 5 seconds with SCS and without SCS. After several repetitions of knee extensions, the patient would show clear signs of fatigue. At that time, the immediate impact of SCS on the patient's maximum voluntary contraction (MVC) during fatigued knee extension is tested. FIGS. 7A-C illustrate the various torque measurements over time. FIG. 7A illustrates single traces of torque produced by the patient with stimulation (see element 704) and without stimulation (see element 702) during maximum voluntary contraction repetitions. In FIG. 7B, each dot corresponds to the mean torque produced during one single trial with and without stimulation. In FIG. 7C, each dot corresponds to the maximum torque produced during one single trial with and without stimulation. In FIGS. 7B and 7C, the squares markers represent the mean across repetitions, and the error bars correspond to the standard error of the mean. As shown, the MVC during SCS stimulation is significantly higher on days 12 and 27.

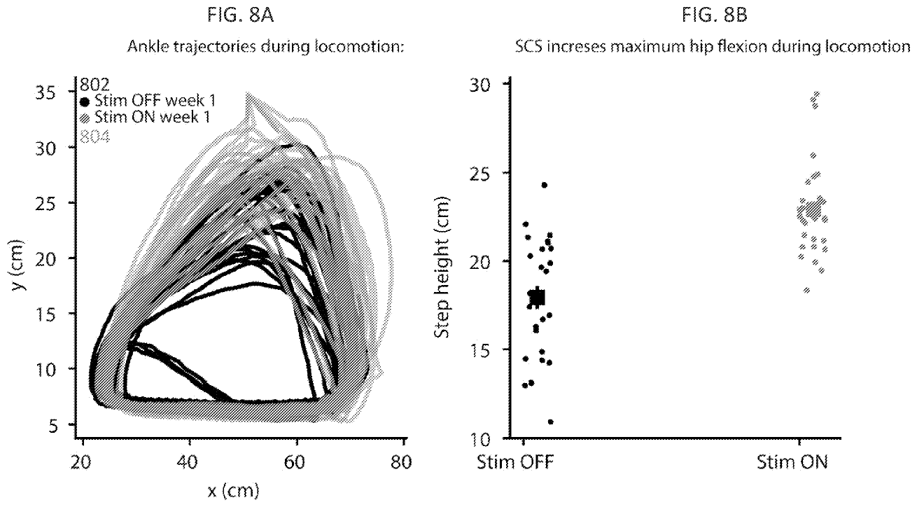

Hip Flexion During Locomotion: The patient is asked to produce maximum hip flexion by raising his knees as high as possible while his body weight is supported by a treadmill. While the patient is walking, periods of stimulation are alternated on and off every 20 seconds. The patient is given no visual feedback of his legs during the task. To measure changes in step height, tracking markers are placed on the ankle, toe and metatarsal on each foot. A machine-learning model (e.g., a deep neural network) can be trained to track the markers. FIGS. 8A-B illustrate that spinal cord stimulation increases maximum hip flexion during locomotion. Specifically, FIG. 8A illustrates traces of an ankle marker during locomotion without stimulation (see element 802) and with stimulation (see element 804). In FIG. 8B, the dots represent the height of each step computed as the difference between the smaller and the higher values of each trace in FIG. 8A. The squares markers represent the mean across steps, and the error bars correspond to the standard error of the mean. As shown in FIGS. 8A-B, SCS has provided an immediate increase in the patient's maximum hip flexion during locomotion, which translates to a higher step height.

Balance: The patient is asked to walk heel-to-toe while staring straight ahead on a beam that becomes narrower with distance, while receiving no visual feedback of his feet. In the first session, the patient is worse with stimulation on and consistently traverses less distance. The patient reports that the stimulation perturbs his balance because the precise control of his legs has become more difficult.

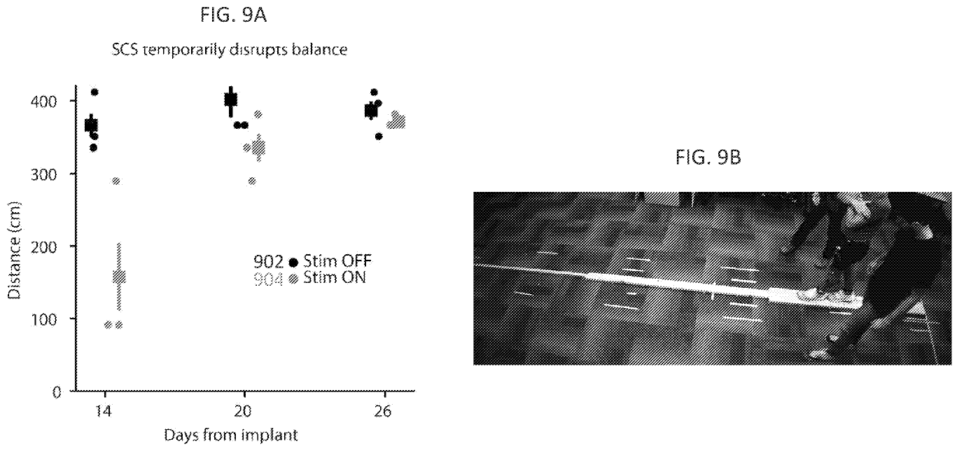

However, when the patient's balance is re-tested during the second session (a week after the first session) and the third sessions (a week after the second session), it is found that the patient has learned to control the stimulation and improved his balance such that stimulation on and off would produce similar distances. FIGS. 9A-B illustrate that spinal cord stimulation on the patient only temporarily disrupts balance. In FIG. 9A, each dot is the walked distance for the patient in the narrowing beam test without stimulation (see element 902) and with stimulation (see element 904). The squares represent the mean and error bars represent the standard error of the mean across repetitions. FIG. 9B is a picture of the patient walking on the narrowing bean.

Sit-to-Stand Transitions: The patient is asked to stand from a position where one knee is on the ground and the opposite foot is planted on the ground. Different knee heights are tested until the maximum height where the patient is unable to stand is identified. The stimulation is then applied. The patient is able to consistently reach a standing position when the right knee is planted on the ground.

The patient is also asked to stand from a sitting position on a box measuring 46 cm from the ground. Without stimulation, the patient is able to perform compensated standing while his legs are exaggeratedly spread out. The patient is then instructed to continue to attempt to stand bringing his feet closer together until a separation distance between his feet is reached where he is no longer able to stand. The stimulation is then applied to the patient with his feet in the same position, i.e., separated at the distance where he was unable to stand, and the patient is able to consistently stand from such position.

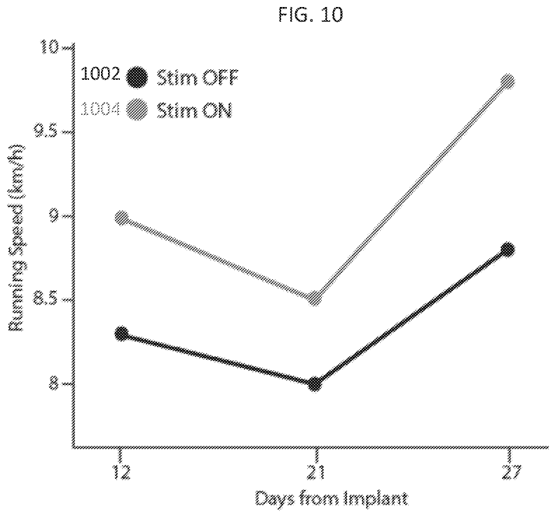

Maximum Speed: The patient is asked to run on the treadmill, and the velocity is progressively increased every 30 seconds until he reported that his maximum velocity is reached. FIG. 10 illustrates that SCS (see element 1004) robustly increases the patient's maximum velocity relative to when SCS is not applied (see element 1002).

### Observed Long-Term Effects

In this example, the long-term effects of SCS are measured in terms of hip flexion during locomotion and maximum voluntary contraction. Each measurement is discussed below. It should be appreciated that different metrics can be used depending on the targeted muscle(s) and conditions of a patient.

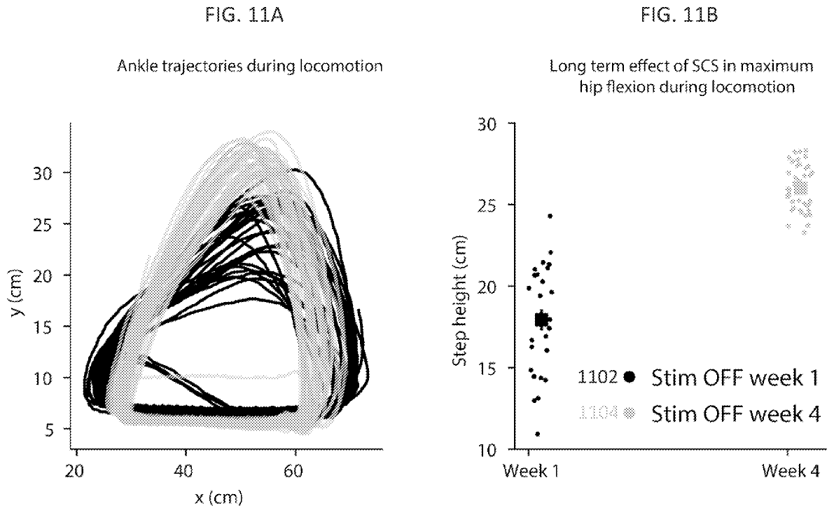

Hip Flexion During Locomotion: The patient is asked to walk on the treadmill while raising his knees as high as possible in week 4. FIGS. 11A-11B illustrate a long-term effect of SCS in hip flexion over a four-week experiment. FIG. 11A illustrates traces of the ankle marker during locomotion, comparing traces from week 1 without stimulation (see element 1102) to traces from week 4 without stimulation (see element 1104). In FIG. 11B, the dots are the height of each step computed as the difference between the smaller and the higher values of each trace in FIG. 11A. The square is the mean and standard error of the mean across steps. By comparing week 1 and week 4, a noticeable increase is observed in the patient's maximum hip flexion during locomotion even without stimulation, indicating a long-term effect of the SCS intervention. Without being bound to any particular theory, application of the electrical stimulus to the patient over time may lead to ion channel remodeling on the MN membrane and a persistent increase in the firing rate probability of spinal MNs which may improve motor function, even in the absence of stimulation.

Maximum Voluntary Contraction: At the beginning of each session with the isokinetic machine, the patient's MVC is measured as torque in Newton-meters (Nm) during knee extension and/or hip flexion without stimulation. The MVC of the patient during knee extension is about 20 Nm, while the MVC of a healthy young adult is above 100 Nm. The patient is asked to produce his maximum torque in these joints for 6 repetitions of 5 seconds each. The knee extension is tested twice per week and hip flexion is tested pre-implant, in week 1, in week 3 and post-explant. In general, significant increases are observed in MVC for all movements starting from week 2.

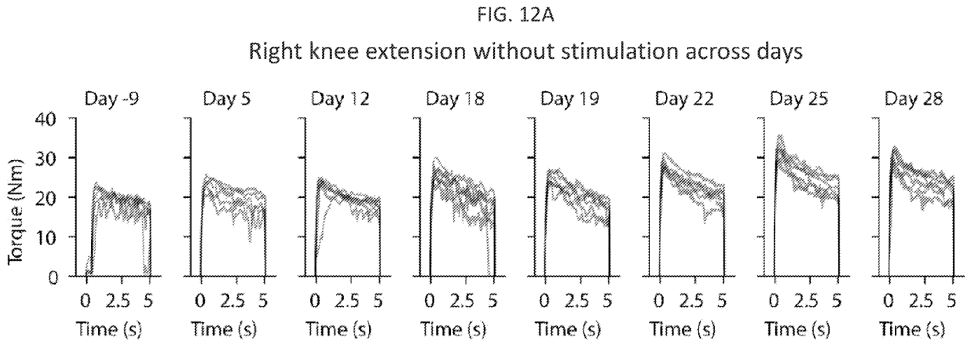

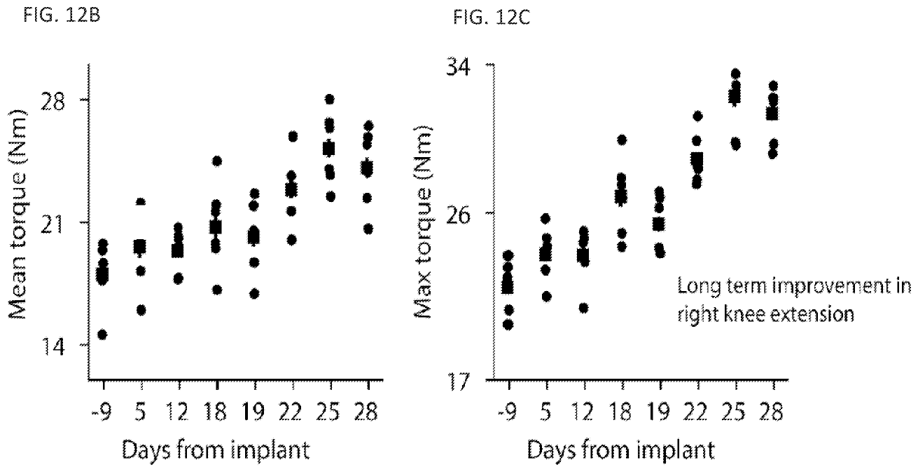

In terms of right knee extension, starting from day 18 to the end of the experiment, a consistent increase in maximum and mean torque is observed when SCS is turned off. The final increase from pre-implantation to the end of study is +43.5%. FIGS. 12A-12C illustrate long-term improvement in right knee extension of the patient. FIG. 12A illustrates torque traces for the 6 repetitions in each session. In FIGS. 12B-C, each dot is the mean torque for each 5-second repetition. The squares are the mean and the standard error of the mean of the mean torque across repetitions. As shown, the effect appears to be linear and there is no evidence of reaching a plateau by week 4. Starting from day 18 to the end of the experiment, the patient experiences a consistent increase in maximum and mean right knee extension torque when SCS is turned off. The final increase from pre-implantation to the end of study is +43.5% for the patient.

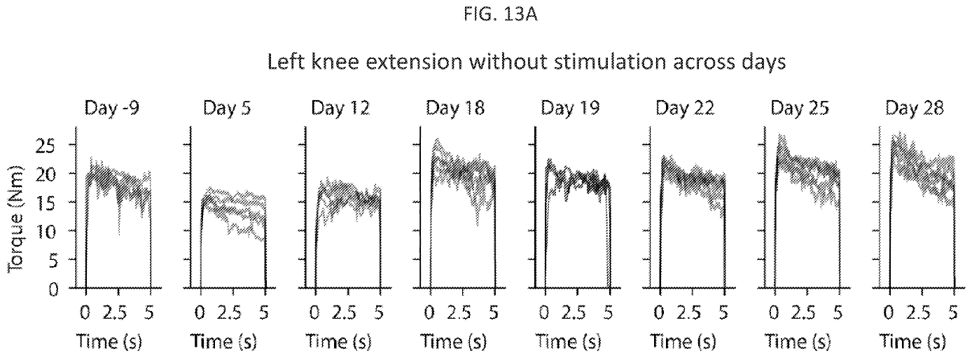

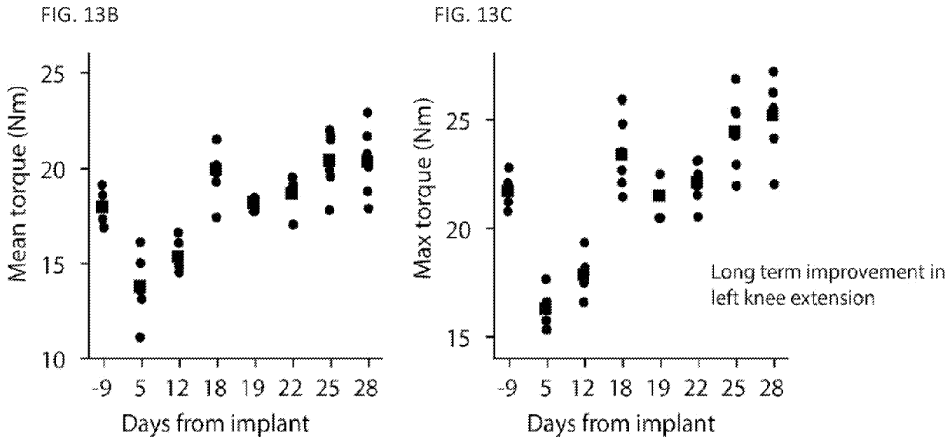

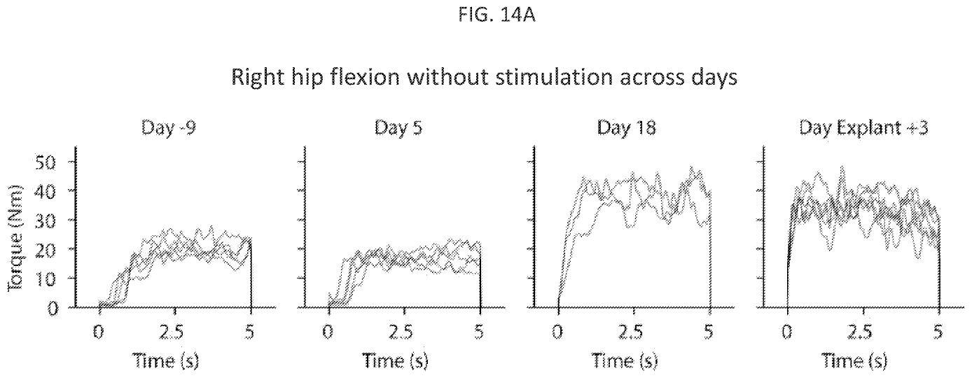

A consistent increase in left knee extension of the patient is similarly observed from day 18 to the end of the four-week experiment, even though it is the patient's more impaired leg. Left knee extension results during the four weeks of SCS experiments are largely consistent with the results for the right knee extension, in that no increase is observed before day 18 and sustained increase is then observed afterwards. The final increase from day 5 to end of study is +65.1%. FIGS. 13A-C illustrate long-term improvement in left knee extension. FIG. 13A illustrates torque traces for the 6 repetitions in each session. In FIGS. 13B-C, each dot is the mean torque for each 5-second repetition. The squares are the mean and the standard error of the mean of the mean torque across repetitions.

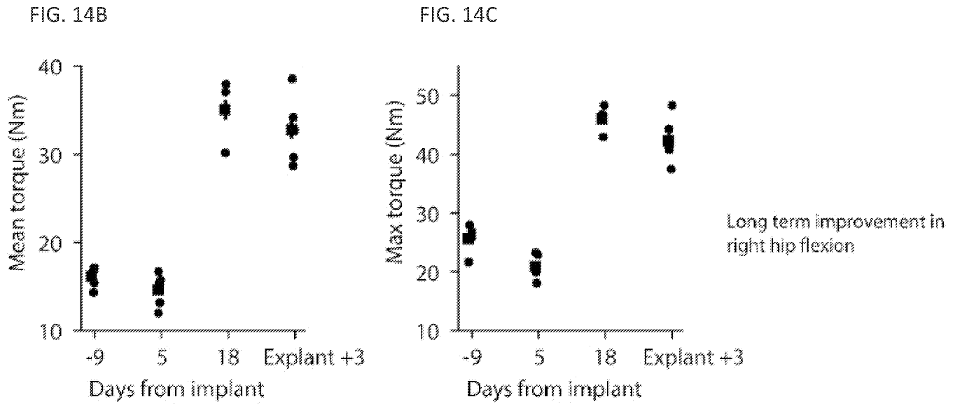

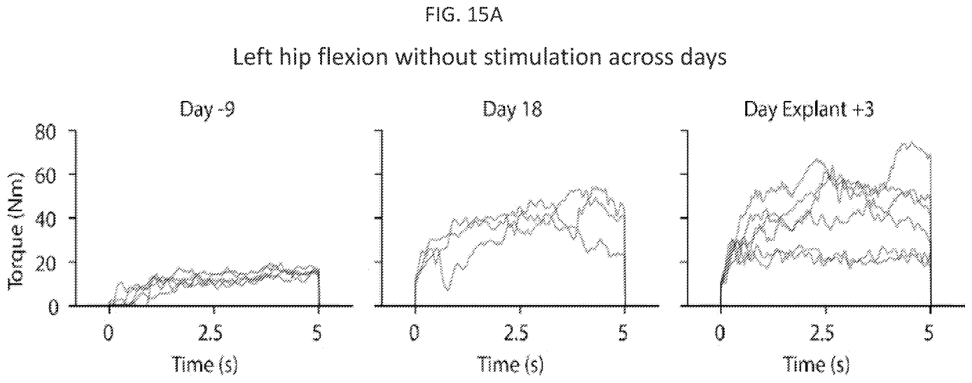

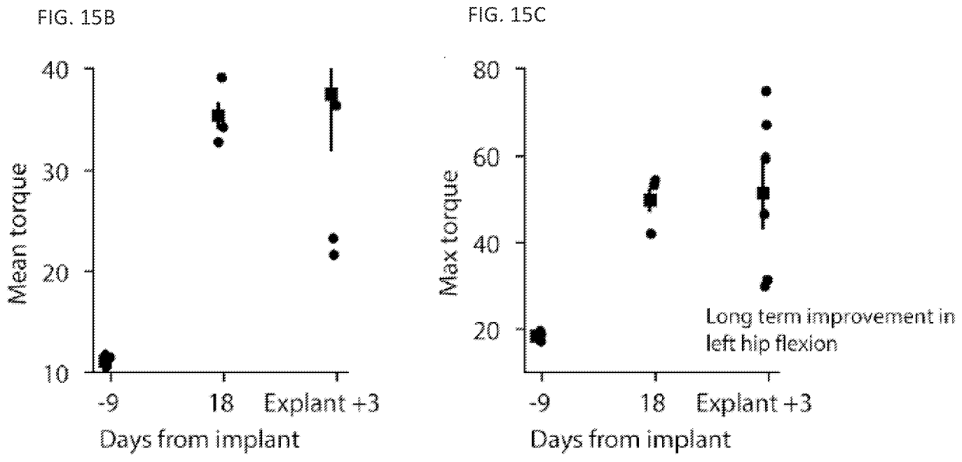

The timing of improvements in right and left hip flexion of the patient is consistent with the results in knee extension, with an increase in both being observed from day 18 to the end of the four-week experiment for hip flexion. The final increase from pre-implant to post-explant is +65.1% on the right and +179.7% on the left for the patient. FIGS. 14A-C illustrate long-term improvement in right hip flexion. FIG. 14A illustrates torque traces for the 6 repetitions in each session. In FIGS. 14B-C, each dot is the mean torque for each 5-second repetition. The squares are the mean and the standard error of the mean of the mean torque across repetitions. FIGS. 15A-C illustrate long-term improvement in left hip flexion for the patient. FIG. 15A illustrates torque traces for the 6 repetitions in each session. In FIGS. 15B-C, each dot is the mean torque for each 5-second repetition. The squares are the mean and the standard error of the mean of the mean torque across repetitions.

As a result of these improvements in hip flexion and knee extension, the patient's overall range of motion is much higher after the end of the experiment than before. The patient's improvements in range of motion are most noticeable in hip flexion and whole body movement. Without being bound to any particular theory, the application of the electrical stimulus to sensory neurons may directly recruit mono- and poly-synaptic excitatory pathways in the spinal cord, which in turn increases the membrane potential and firing rate probability of spinal MNs innervating the body region of the patient with the motor impairment due to SMA. This recruitment of pathways may increase neuroplasticity, allowing the patient to improve motor function.

### Clinical Outcome Tests

Clinical outcome tests are administered, including the manual muscle test, the 6-minute walk test and the leg circumference test, and immediate and long-term effects are observed across the clinical outcome tests, as discussed below.

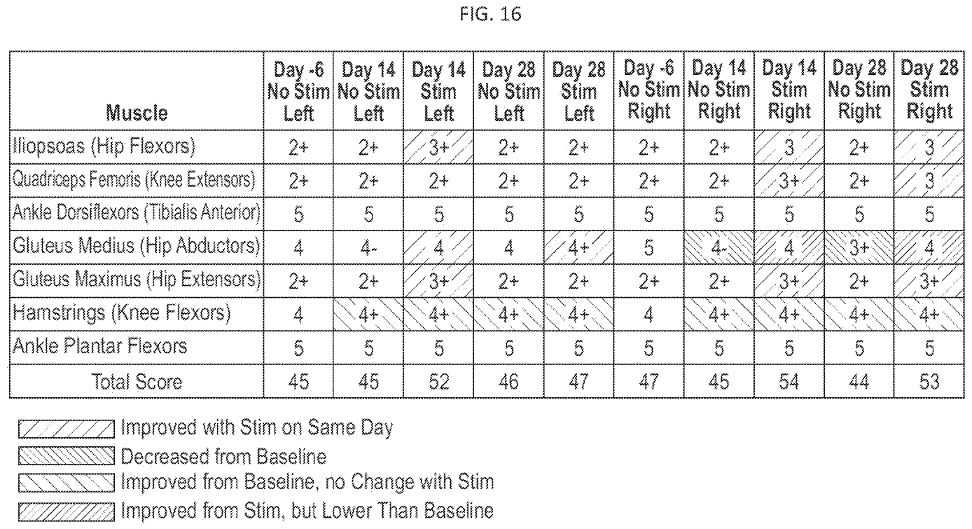

Manual Muscle Test: The manual muscle test (MMT) is a commonly accepted method of evaluating muscle strength. It individually measures the strength of each muscle and then sums them up to report a total score. The MMT is performed on the patient both pre-implant and on days 14 and 28 after implantation. The MNT shows consistent immediate effects from stimulation. Specifically, both the knee extensors/flexors and hip extensors/flexors show improved scores with stimulation, as shown in the spreadsheet illustrated by FIG. 16, which contains manual muscle test scores for an SMA patient by muscle and session with and without stimulation.

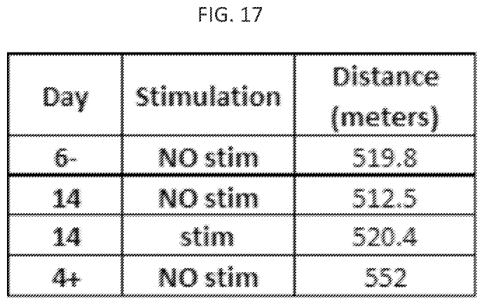

6-minute Walk Test: The 6-minute walk test is performed on the patient pre-implant, on day 14 with and without stimulation and after explantation of the electrodes. No significant immediate effects from stimulation are observed, but large improvements from the long term effects of stimulation in total distance traveled is observed for the patient, as shown in the table illustrated by FIG. 17. FIG. 17 displays distances traveled by the patient during a six-minute walk test across different sessions comparing stimulation off vs. stimulation on.

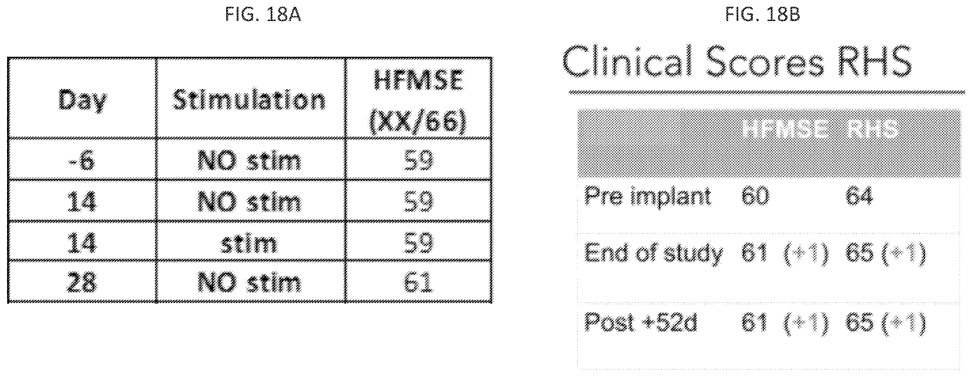

Clinical Scales, Hammersmith Functional Motor Scale Expanded (HFMSE) and Revised Hammersmith Scale (RHS): The HFMSE and RHS tests are validated instruments to assess the motor ability of children and adults with SMA Types 2 and 3. The results of HFMSE and RHS tests are measured before implant, at the date of explant, and at a follow-up post-explant. The HFMSE test for the patient shows small but relevant long-term effects with an increase in total score, achieving a final HFMSE score of 61. Improvements in the HFMSE can be generally very difficult to achieve for patients. Thus, even small changes (1-2 points in the present case) are indicative of improvements, as shown in the tables illustrated by FIGS. 18A and 18B, which contain HFMSE scores for the patient. Likewise, as shown in the table illustrated by FIG. 18B, the RHS test for the patient shows a pre-implant RHS score of 64 and an explant RHS score of 65 (a 1 point increase). The increase in RHS score remains consistent 52 days after explant, which is indicative of small but relevant long-term effects associated with the four-week experiment.

Leg Circumference: On day 18 after implantation, circumferences of the patient's left and right legs are measured from 15.24 cm above the left and right patella, respectively. The patient's left and right leg circumferences are measured to be 43.5 and 46 cm, respectively. On day 5 after the end of the four-week experiment and explantation, the left and right leg circumferences of the patient are measured to be 44 and 45 cm, respectively. These small differences may be considered insignificant.

Additional details of the clinical trial can be found with the Clinicaltrials.gov Identifier NCT05430113, “Spinal Cord Stimulation in Spinal Muscular Atrophy (SCSinSMA),” available at https://clinicaltrials.gov/ct2/show/NCT05430113.

The disclosure will now be further described by the following numbered embodiments which are to be read in connection with the preceding paragraphs, and which do not limit the disclosure. The features, options and preferences as described above apply also to the following embodiments.

Embodiment 1. A method for treating spinal muscular atrophy (SMA) in a subject, comprising: applying a therapeutically effective amount of an electrical stimulus to sensory neurons innervating a body region of the subject with a motor impairment due to SMA, wherein the electrical stimulus is applied with one or more electrodes controlled by a neurostimulator, and wherein application of the electrical stimulus treats the motor impairment due to SMA in the subject.

Embodiment 2. The method of Embodiment 1, wherein applying the electrical stimulus increases the firing rate probability of spinal motoneurons innervating the body region of the subject with the motor impairment due to SMA.

Embodiment 3. The method of Embodiment 1 or Embodiment 2, wherein the stimulation is applied at or below a motor threshold such that the stimulation does not directly elicit movement and/or muscle activity of the body region of the subject with the motor impairment due to SMA.

Embodiment 4. The method of any one of Embodiments 1-3, wherein the electrical stimulus comprises electrical pulses having an amplitude of about 10 μA to about 100 mA, a width between about 40 μs and about 2 ms, and a frequency of about 10 Hz to about 2000 Hz.

Embodiment 5. The method of any one of Embodiments 1-4, wherein the electrical stimulus comprises electrical pulses having an amplitude of about 10 μA to about 10 mA, a width between about 40 μs and about 2 ms, and a frequency of about 10 Hz to about 1000 Hz.

Embodiment 6. The method of any one of Embodiments 1-5, wherein the electrical stimulus comprises electrical pulses having an amplitude of about 100 μA to about 10 mA, a width between about 40 μs and about 500 μs, and a frequency of about 10 Hz to about 1000 Hz.

Embodiment 7. The method of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 8. The method of Embodiment 7, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 9. The method of any one of Embodiments 4-8, wherein the pulses are cathodic-first biphasic or monophasic charge balanced pulses.

Embodiment 10. The method of any one of the prior Embodiments, wherein electrical stimulation is applied for at least 2 hours/day over a period of at least 6 months.

Embodiment 11. The method of any one of Embodiments 1-9, wherein electrical stimulation is applied for at least 1 hour/day over a period of at least 1 month.

Embodiment 12. The method of any one of the prior Embodiments, wherein the one or more electrodes are contained within an array of independently controllable electrodes implanted in the subject.

Embodiment 13. The method of Embodiment 12, wherein the electrode array is a multi-electrode paddle array.

Embodiment 14. The method of any one of the prior Embodiments, wherein the one or more electrodes are implanted epidurally at the spinal cord of the subject.

Embodiment 15. The method of any one of Embodiments 1-14, wherein the one or more electrodes are implanted at the dorsal rootlets of the one or more sensory neurons innervating the body region with the motor impairment of the subject.

Embodiment 16. The method of any one of Embodiments 1-14, wherein the one or more electrodes are implanted at the dorsolateral aspect of the spinal cord adjacent to the dorsal roots of the one or more sensory neurons innervating the body region with the motor impairment of the subject.

Embodiment 17. The method of any one of Embodiments 1-13, wherein the one or more electrodes are implanted at the dorsal root ganglia of the one or more sensory neurons innervating the body region with the motor impairment of the subject. Embodiment 18. The method of any one of Embodiments 1-12, wherein the one or more electrodes are contained in a cuff that surrounds a peripheral nerve containing the sensory neurons innervating the body region of the subject with the motor impairment due to SMA.

Embodiment 19. The method of any one of Embodiments 1-12, wherein the one or more electrodes penetrate a peripheral nerve or dorsal root ganglion containing the sensory neurons innervating the body region of the subject with the motor impairment due to SMA.

Embodiment 20. The method of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 21. The method of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 22. The method of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 23. The method of any one of the prior Embodiments, wherein the neurostimulator is an external or implanted pulse generator.

Embodiment 24. The method of any one of the prior Embodiments, further comprising implanting the neurostimulator in the subject.

Embodiment 25. The method of any one of the prior Embodiments, further comprising selecting the subject with SMA for treatment.

Embodiment 26. The method of any one of the prior Embodiments, wherein the motor impairment comprises partial or complete paralysis, loss of dexterity, loss of muscle strength, and/or uncontrollable muscle tone.

Embodiment 27. The method of any one of the prior Embodiments, wherein the locations of the one or more electrodes are chosen based on the body region of the subject with the motor impairment due to SMA.

Embodiment 28. The method of Embodiment 27, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 29. The method of any one of the prior Embodiments, wherein at least one of the one or more electrodes is placed transcutaneously.

Embodiment 30. The method of any one of the prior Embodiments, wherein at least one of the one or more electrodes is implanted.

Embodiment 31. A method of stimulating one or more motoneurons impaired by SMA, said method comprising applying an electrical stimulus to at least one of said motoneurons wherein said motoneurons innervate a body region of a subject with a motor impairment due to SMA.

Embodiment 32. A method for treating spinal muscular atrophy (SMA) in a subject, comprising applying a therapeutically effective amount of an electrical stimulus to sensory neurons innervating a body region of the subject with a motor impairment due to SMA.

Embodiment 33. A method of increasing firing rate of motoneurons impaired by SMA in a subject, comprising applying a therapeutically effective amount of an electrical stimulus to sensory neurons innervating a body region of the subject to increase the motoneuron firing rate of the subject.

Embodiment 34. The method of Embodiment 33, wherein the subject has a first motoneuron firing rate before the application of the therapeutically effective amount of the electrical stimulus and a second motoneuron firing rate after the application.

Embodiment 35. The method of Embodiment 33 or Embodiment 34, wherein the electrical stimulus comprises electrical pulses having an amplitude of about 10 μA to about 100 mA, a width between about 40 μs and about 2 ms, and a frequency of about 10 Hz to about 2000 Hz.

Embodiment 36. The method of any one of Embodiments 33-35, wherein the electrical stimulus is applied for at least 1 hour/day over a period of at least 1 month. Embodiment 37. The method of any one of Embodiments 33-36, wherein the motoneuron firing rate of the subject is computed from electrical signals produced by the motoneurons while varying the parameters of the electrical stimulus.

Embodiment 38. The method of any one of Embodiments 33-37, wherein increasing firing rate of motoneurons impaired by SMA increases joint torques and muscle strength of the subject.

Embodiment 39. A method of increasing excitability of motoneurons impaired by SMA in a subject to a sensory afferent input, comprising applying a therapeutically effective amount of an electrical stimulus to sensory neurons innervating a body region of the subject to increase the motoneuron excitability of the subject.

Embodiment 40. The method of Embodiment 39, wherein the subject has a first motoneuron excitability before the application of the therapeutically effective amount of the electrical stimulus and a second motoneuron excitability after the application.

Embodiment 41. The method of Embodiment 39 or Embodiment 40, wherein the electrical stimulus comprises electrical pulses having an amplitude of about 10 μA to about 100 mA, a width between about 40 μs and about 2 ms, and a frequency of about 10 Hz to about 2000 Hz.

Embodiment 42. The method of any one of Embodiments 39-41, wherein the electrical stimulus is applied for at least 1 hour/day over a period of at least 1 month.

Embodiment 43. The method of any one of Embodiments 39-42, wherein the motoneuron excitability of the subject is computed from electrical signals produced by the motoneurons while varying the parameters of the electrical stimulus.

Embodiment 44. The method of any one of Embodiments 39-43, wherein increasing excitability of motoneurons impaired by SMA increases joint torques and muscle strength of the subject.

Embodiment 45. A therapeutically effective amount of an electrical stimulus to sensory neurons innervating a body region of the subject with a motor impairment due to SMA for treating SMA in a subject, wherein the electrical stimulus is to be applied with one or more electrodes controlled by a neurostimulator, and wherein application of the electrical stimulus treats the motor impairment due to SMA in the subject.

Embodiment 46. The electrical stimulus for use of Embodiment 45, wherein applying the electrical stimulus increases the firing rate probability of spinal motoneurons innervating the body region of the subject with the motor impairment due to SMA.

Embodiment 47. The electrical stimulus for use of Embodiment 45 or Embodiment 46, wherein the stimulation is applied at or below a motor threshold such that the stimulation does not directly elicit movement and/or muscle activity of the body region of the subject with the motor impairment due to SMA.

Embodiment 48. The electrical stimulus for use of Embodiments 45-47, wherein the electrical stimulus comprises electrical pulses having an amplitude of about 10 μA to about 100 mA, a width between about 40 μs and about 2 ms, and a frequency of about 10 Hz to about 2000 Hz.

Embodiment 49. The electrical stimulus for use of Embodiments 45-48, wherein the electrical stimulus comprises electrical pulses having an amplitude of about 10 μA to about 10 mA, a width between about 40 μs and about 2 ms, and a frequency of about 10 Hz to about 1000 Hz.

Embodiment 50. The electrical stimulus for use of Embodiments 45-49, wherein the electrical stimulus comprises electrical pulses having an amplitude of about 100 μA to about 10 mA, a width between about 40 μs and about 500 μs, and a frequency of about 10 Hz to about 1000 Hz.

Embodiment 51. The electrical stimulus for use of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 52. The electrical stimulus for use of Embodiment 51, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 53. The electrical stimulus for use of any one of Embodiments 48-52, wherein the pulses are cathodic-first biphasic or monophasic charge balanced pulses.

Embodiment 54. The electrical stimulus for use of any one of the prior Embodiments, wherein the electrical stimulus is applied for at least 2 hours/day over a period of at least 6 months.

Embodiment 55. The electrical stimulus for use of any one of Embodiments 45-53, wherein the electrical stimulus is applied for at least 1 hour/day over a period of at least 1 month.

Embodiment 56. The electrical stimulus for use of any one of the prior Embodiments, wherein the one or more electrodes are contained within an array of independently controllable electrodes implanted in the subject.

Embodiment 57. The electrical stimulus for use of Embodiment 56, wherein the electrode array is a multi-electrode paddle array.

Embodiment 58. The electrical stimulus for use of any one of the prior Embodiments, wherein the one or more electrodes are implanted epidurally at the spinal cord of the subject.

Embodiment 59. The electrical stimulus for use of any one of Embodiments 45-58, wherein the one or more electrodes are implanted at the dorsal rootlets of the one or more sensory neurons innervating the body region with the motor impairment of the subject.

Embodiment 60. The electrical stimulus for use of any one of Embodiments 45-58, wherein the one or more electrodes are implanted at the dorsolateral aspect of the spinal cord adjacent to the dorsal roots of the one or more sensory neurons innervating the body region with the motor impairment of the subject.

Embodiment 61. The electrical stimulus for use of any one of Embodiments 45-57, wherein the one or more electrodes are implanted at the dorsal root ganglia of the one or more sensory neurons innervating the body region with the motor impairment of the subject.

Embodiment 62. The electrical stimulus for use of any one of Embodiments 45-56, wherein the one or more electrodes are contained in a cuff that surrounds a peripheral nerve containing the sensory neurons innervating the body region of the subject with the motor impairment due to SMA.

Embodiment 63. The electrical stimulus for use of any one of Embodiments 45-56, wherein the one or more electrodes penetrate a peripheral nerve or dorsal root ganglion containing the sensory neurons innervating the body region of the subject with the motor impairment due to SMA.

Embodiment 64. The electrical stimulus for use of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 65. The electrical stimulus for use of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 66. The electrical stimulus for use of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 67. The electrical stimulus for use of any one of the prior Embodiments, wherein the neurostimulator is an external or implanted pulse generator.

Embodiment 68. The electrical stimulus for use of any one of the prior Embodiments, wherein the neurostimulator is implanted in the subject.

Embodiment 69. The electrical stimulus for use of any one of the prior Embodiments, wherein the subject with SMA is selected for treatment.

Embodiment 70. The electrical stimulus for use of any one of the prior Embodiments, wherein the motor impairment comprises partial or complete paralysis, loss of dexterity, loss of muscle strength, and/or uncontrollable muscle tone.

Embodiment 71. The electrical stimulus for use of any one of the prior Embodiments, wherein the locations of the one or more electrodes are chosen based on the body region of the subject with the motor impairment due to SMA.

Embodiment 72. The electrical stimulus for use of Embodiment 71, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 73. The electrical stimulus for use of any one of the prior Embodiments, wherein at least one of the one or more electrodes is placed transcutaneously.

Embodiment 74. The electrical stimulus for use of any one of the prior Embodiments, wherein at least one of the one or more electrodes is implanted.

Embodiment 75. Use of one or more electrodes controlled by a neurostimulator in the manufacture of a medicament for treating SMA in a subject, wherein the one or more electrodes controlled by the neurostimulator are configured to apply a therapeutically effective amount of an electrical stimulus to sensory neurons innervating a body region of the subject with a motor impairment due to SMA.

Embodiment 76. The use of Embodiment 75, wherein applying the electrical stimulus increases the firing rate probability of spinal motoneurons innervating the body region of the subject with the motor impairment due to SMA.

Embodiment 77. The use of Embodiment 75 or Embodiment 76, wherein the stimulation is applied at or below a motor threshold such that the stimulation does not directly elicit movement and/or muscle activity of the body region of the subject with the motor impairment due to SMA.

Embodiment 78. The use of Embodiments 75-77, wherein the electrical stimulus comprises electrical pulses having an amplitude of about 10 μA to about 100 mA, a width between about 40 μs and about 2 ms, and a frequency of about 10 Hz to about 2000 Hz.

Embodiment 79. The use of Embodiments 75-78, wherein the electrical stimulus comprises electrical pulses having an amplitude of about 10 μA to about 10 mA, a width between about 40 μs and about 2 ms, and a frequency of about 10 Hz to about 1000 Hz.

Embodiment 80. The use of Embodiments 75-79, wherein the electrical stimulus comprises electrical pulses having an amplitude of about 100 μA to about 10 mA, a width between about 40 μs and about 500 μs, and a frequency of about 10 Hz to about 1000 Hz.

Embodiment 81. The use of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 82. The use of Embodiment 81, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 83. The use of any one of Embodiments 78-82, wherein the pulses are cathodic-first biphasic or monophasic charge balanced pulses.

Embodiment 84. The use of any one of the prior Embodiments, wherein the electrical stimulus is applied for at least 2 hours/day over a period of at least 6 months.

Embodiment 85. The use of any one of Embodiments 75-83, wherein the electrical stimulus is applied for at least 1 hour/day over a period of at least 1 month.

Embodiment 86. The use of any one of the prior Embodiments, wherein the one or more electrodes are contained within an array of independently controllable electrodes implanted in the subject.

Embodiment 87. The use of Embodiment 86, wherein the electrode array is a multi-electrode paddle array.

Embodiment 88. The use of any one of the prior Embodiments, wherein the one or more electrodes are implanted epidurally at the spinal cord of the subject.

Embodiment 89. The use of any one of Embodiments 75-88, wherein the one or more electrodes are implanted at the dorsal rootlets of the one or more sensory neurons innervating the body region with the motor impairment of the subject.

Embodiment 90. The use of any one of Embodiments 75-88, wherein the one or more electrodes are implanted at the dorsolateral aspect of the spinal cord adjacent to the dorsal roots of the one or more sensory neurons innervating the body region with the motor impairment of the subject.

Embodiment 91. The use of any one of Embodiments 75-87, wherein the one or more electrodes are implanted at the dorsal root ganglia of the one or more sensory neurons innervating the body region with the motor impairment of the subject.

Embodiment 92. The use of any one of Embodiments 75-86, wherein the one or more electrodes are contained in a cuff that surrounds a peripheral nerve containing the sensory neurons innervating the body region of the subject with the motor impairment due to SMA.

Embodiment 93. The use of any one of Embodiments 75-86, wherein the one or more electrodes penetrate a peripheral nerve or dorsal root ganglion containing the sensory neurons innervating the body region of the subject with the motor impairment due to SMA.

Embodiment 94. The use of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 95. The use of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 96. The use of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 97. The use of any one of the prior Embodiments, wherein the neurostimulator is an external or implanted pulse generator.

Embodiment 98. The use of any one of the prior Embodiments, wherein the neurostimulator is implanted in the subject.

Embodiment 99. The use of any one of the prior Embodiments, wherein the subject with SMA is selected for treatment.

Embodiment 100. The use of any one of the prior Embodiments, wherein the motor impairment comprises partial or complete paralysis, loss of dexterity, loss of muscle strength, and/or uncontrollable muscle tone.

Embodiment 101. The use of any one of the prior Embodiments, wherein the locations of the one or more electrodes are chosen based on the body region of the subject with the motor impairment due to SMA.

Embodiment 102. The use of Embodiment 101, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 103. The use of any one of the prior Embodiments, wherein at least one of the one or more electrodes is placed transcutaneously.

Embodiment 104. The use of any one of the prior Embodiments, wherein at least one of the one or more electrodes is implanted.

Embodiment 105. Use of one or more electrodes controlled by a neurostimulator in the manufacture of a medicament for treating SMA in a subject, wherein an electrical stimulus is to be applied using the one or more electrodes controlled by the neurostimulator to sensory neurons innervating a body region of the subject with a motor impairment due to SMA.

Embodiment 106. The use of Embodiment 105, wherein applying the electrical stimulus increases the firing rate probability of spinal motoneurons innervating the body region of the subject with the motor impairment due to SMA.

Embodiment 107. The use of Embodiment 105 or Embodiment 106, wherein the stimulation is applied at or below a motor threshold such that the stimulation does not directly elicit movement and/or muscle activity of the body region of the subject with the motor impairment due to SMA.

Embodiment 108. The use of Embodiments 105-107, wherein the electrical stimulus comprises electrical pulses having an amplitude of about 10 μA to about 100 mA, a width between about 40 μs and about 2 ms, and a frequency of about 10 Hz to about 2000 Hz.

Embodiment 109. The use of Embodiments 105-108, wherein the electrical stimulus comprises electrical pulses having an amplitude of about 10 μA to about 10 mA, a width between about 40 μs and about 2 ms, and a frequency of about 10 Hz to about 1000 Hz.

Embodiment 110. The use of Embodiments 105-109, wherein the electrical stimulus comprises electrical pulses having an amplitude of about 100 μA to about 10 mA, a width between about 40 μs and about 500 μs, and a frequency of about 10 Hz to about 1000 Hz.

Embodiment 111. The use of any one of the prior Embodiments, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

Embodiment 112. The use of Embodiment 111, wherein the stimulation pattern is a series of 3 pulses separated by inter-pulse intervals of about 5 ms, wherein the series is repeated at a frequency of about 30 to about 100 Hz, and wherein the pulse width is about 200 μs.

Embodiment 113. The use of any one of Embodiments 108-112, wherein the pulses are cathodic-first biphasic or monophasic charge balanced pulses.

Embodiment 114. The use of any one of the prior Embodiments, wherein the electrical stimulus is applied for at least 2 hours/day over a period of at least 6 months.

Embodiment 115. The use of any one of Embodiments 105-113, wherein the electrical stimulus is applied for at least 1 hour/day over a period of at least 1 month.

Embodiment 116. The use of any one of the prior Embodiments, wherein the one or more electrodes are contained within an array of independently controllable electrodes implanted in the subject.

Embodiment 117. The use of Embodiment 116, wherein the electrode array is a multi-electrode paddle array.

Embodiment 118. The use of any one of the prior Embodiments, wherein the one or more electrodes are implanted epidurally at the spinal cord of the subject.

Embodiment 119. The use of any one of Embodiments 105-118, wherein the one or more electrodes are implanted at the dorsal rootlets of the one or more sensory neurons innervating the body region with the motor impairment of the subject.

Embodiment 120. The use of any one of Embodiments 105-118, wherein the one or more electrodes are implanted at the dorsolateral aspect of the spinal cord adjacent to the dorsal roots of the one or more sensory neurons innervating the body region with the motor impairment of the subject.

Embodiment 121. The use of any one of Embodiments 105-117, wherein the one or more electrodes are implanted at the dorsal root ganglia of the one or more sensory neurons innervating the body region with the motor impairment of the subject.

Embodiment 122. The use of any one of Embodiments 105-116, wherein the one or more electrodes are contained in a cuff that surrounds a peripheral nerve containing the sensory neurons innervating the body region of the subject with the motor impairment due to SMA.

Embodiment 123. The use of any one of Embodiments 105-116, wherein the one or more electrodes penetrate a peripheral nerve or dorsal root ganglion containing the sensory neurons innervating the body region of the subject with the motor impairment due to SMA.

Embodiment 124. The use of any one of the prior Embodiments, wherein the body region of the subject is selected from at least one of lower back, hip, leg, ankle, and foot; and the one or more electrodes are implanted at the dorsolateral aspect of the spinal cord and span one or more of the T11-S1 nerve roots.

Embodiment 125. The use of any one of the prior Embodiments, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

Embodiment 126. The use of any one of the prior Embodiments, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

Embodiment 127. The use of any one of the prior Embodiments, wherein the neurostimulator is an external or implanted pulse generator.

Embodiment 128. The use of any one of the prior Embodiments, wherein the neurostimulator is implanted in the subject.

Embodiment 129. The use of any one of the prior Embodiments, wherein the subject with SMA is selected for treatment.

Embodiment 130. The use of any one of the prior Embodiments, wherein the motor impairment comprises partial or complete paralysis, loss of dexterity, loss of muscle strength, and/or uncontrollable muscle tone.

Embodiment 131. The use of any one of the prior Embodiments, wherein the locations of the one or more electrodes are chosen based on the body region of the subject with the motor impairment due to SMA.

Embodiment 132. The use of Embodiment 131, wherein the locations of the one or more electrodes are determined by: stimulating a plurality of contact points in the subject to target at least one muscle within the body region of the subject with the motor impairment due to SMA; measuring the electrical activity associated with said muscle in response to the stimulation; and identifying one or more contact points in the plurality of contact points as locations of the electrodes based on said electrical activity.

Embodiment 133. The use of any one of the prior Embodiments, wherein at least one of the one or more electrodes is placed transcutaneously.

Embodiment 134. The use of any one of the prior Embodiments, wherein at least one of the one or more electrodes is implanted.

The foregoing description, for purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

It will be apparent that the precise details of the methods or compositions described may be varied or modified without departing from the spirit of the described implementations. All such modifications and variations that fall within the scope and spirit of the claims below are claimed.

It is claimed:

1. A method for treating spinal muscular atrophy (SMA) in a subject, comprising:
applying a therapeutically effective amount of an electrical stimulus to sensory neurons innervating a body region of the subject with a motor impairment due to SMA, wherein the electrical stimulus is applied with one or more electrodes controlled by a neurostimulator, and wherein application of the electrical stimulus treats the motor impairment due to SMA in the subject.

2. The method of claim 1, wherein applying the electrical stimulus increases the firing rate probability of spinal motoneurons innervating the body region of the subject with the motor impairment due to SMA.

3. The method of claim 1 or claim 2, wherein the stimulation is applied at or below a motor threshold such that the stimulation does not directly elicit movement and/or muscle activity of the body region of the subject with the motor impairment due to SMA.

4. The method of any one of claims 1-3, wherein the electrical stimulus comprises electrical pulses having an amplitude of about 10 μA to about 100 mA, a width between about 40 μs and about 2 ms, and a frequency of about 10 Hz to about 2000 Hz.

5. The method of any one of claims 1-4, wherein the electrical stimulus comprises electrical pulses having an amplitude of about 10 μA to about 10 mA, a width between about 40 μs and about 2 ms, and a frequency of about 10 Hz to about 1000 Hz.

6. The method of any one of claims 1-5, wherein the electrical stimulus comprises electrical pulses having an amplitude of about 100 μA to about 10 mA, a width between about 40 μs and about 500 μs, and a frequency of about 10 Hz to about 1000 Hz.

7. The method of any one of the prior claims, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

8. The method of claim 7, wherein the stimulation pattern is a series of 3 pulses separated by inter-pulse intervals of about 5 ms, wherein the series is repeated at a frequency of about 30 to about 100 Hz, and wherein the pulse width is about 200 μs.

9. The method of any one of claims 4-8, wherein the pulses are cathodic-first biphasic or monophasic charge balanced pulses.

10. The method of any one of the prior claims, wherein the electrical stimulus is applied for at least 2 hours/day over a period of at least 6 months.

11. The method of any one of claims 1-9, wherein the electrical stimulus is applied for at least 1 hour/day over a period of at least 1 month.

12. The method of any one of the prior claims, wherein the one or more electrodes are contained within an array of independently controllable electrodes implanted in the subject.

13. The method of claim 12, wherein the electrode array is a multi-electrode paddle array.

14. The method of any one of the prior claims, wherein the one or more electrodes are implanted epidurally at the spinal cord of the subject.

15. The method of any one of claims 1-14, wherein the one or more electrodes are implanted at the dorsal rootlets of the one or more sensory neurons innervating the body region with the motor impairment of the subject.

16. The method of any one of claims 1-14, wherein the one or more electrodes are implanted at the dorsolateral aspect of the spinal cord adjacent to the dorsal roots of the one or more sensory neurons innervating the body region with the motor impairment of the subject.

17. The method of any one of claims 1-13, wherein the one or more electrodes are implanted at the dorsal root ganglia of the one or more sensory neurons innervating the body region with the motor impairment of the subject.

18. The method of any one of claims 1-12, wherein the one or more electrodes are contained in a cuff that surrounds a peripheral nerve containing the sensory neurons innervating the body region of the subject with the motor impairment due to SMA.

19. The method of any one of claims 1-12, wherein the one or more electrodes penetrate a peripheral nerve or dorsal root ganglion containing the sensory neurons innervating the body region of the subject with the motor impairment due to SMA.

20. The method of any one of the prior claims, wherein the body region of the subject is selected from at least one of lower back, hip, leg, ankle, and foot; and the one or more electrodes are implanted at the dorsolateral aspect of the spinal cord and span one or more of the T11-S1 nerve roots.

21. The method of any one of the prior claims, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

22. The method of any one of the prior claims, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

23. The method of any one of the prior claims, wherein the neurostimulator is an external or implanted pulse generator.

24. The method of any one of the prior claims, further comprising implanting the neurostimulator in the subject.

25. The method of any one of the prior claims, further comprising selecting the subject with SMA for treatment.

26. The method of any one of the prior claims, wherein the motor impairment comprises partial or complete paralysis, loss of dexterity, loss of muscle strength, and/or uncontrollable muscle tone.

27. The method of any one of the prior claims, wherein the locations of the one or more electrodes are chosen based on the body region of the subject with the motor impairment due to SMA.

28. The method of claim 27, wherein the locations of the one or more electrodes are determined by:
stimulating a plurality of contact points in the subject to target at least one muscle within the body region of the subject with the motor impairment due to SMA;
measuring the electrical activity associated with said muscle in response to the stimulation; and
identifying one or more contact points in the plurality of contact points as locations of the electrodes based on said electrical activity.

29. The method of any one of the prior claims, wherein at least one of the one or more electrodes is placed transcutaneously.

30. The method of any one of the prior claims, wherein at least one of the one or more electrodes is implanted.

31. A method of stimulating one or more motoneurons impaired by SMA, said method comprising applying an electrical stimulus to at least one of said motoneurons, wherein said motoneurons innervate a body region of a subject with a motor impairment due to SMA.

32. A method for treating SMA in a subject, comprising applying a therapeutically effective amount of an electrical stimulus to sensory neurons innervating a body region of the subject with a motor impairment due to SMA.

33. A method of increasing firing rate of motoneurons impaired by SMA in a subject, comprising applying a therapeutically effective amount of an electrical stimulus to sensory neurons innervating a body region of the subject to increase the motoneuron firing rate of the subject.

34. The method of claim 33, wherein the subject has a first motoneuron firing rate before the application of the therapeutically effective amount of the electrical stimulus and a second motoneuron firing rate after the application.

35. The method of claim 33 or claim 34, wherein the electrical stimulus comprises electrical pulses having an amplitude of about 10 μA to about 100 mA, a width between about 40 μs and about 2 ms, and a frequency of about 10 Hz to about 2000 Hz.

36. The method of any one of claims 33-35, wherein the electrical stimulus is applied for at least 1 hour/day over a period of at least 1 month.

37. The method of any one of claims 33-36, wherein the motoneuron firing rate of the subject is computed based on electrical signals produced by the motoneurons while varying the parameters of the electrical stimulus.

38. The method of any one of claims 33-37, wherein increasing firing rate of motoneurons impaired by SMA increases joint torques and muscle strength of the subject.

39. A method of increasing excitability of motoneurons impaired by SMA in a subject to a sensory afferent input, comprising applying a therapeutically effective amount of an electrical stimulus to sensory neurons innervating a body region of the subject to increase the motoneuron excitability of the subject.

40. The method of claim 39, wherein the subject has a first motoneuron excitability before the application of the therapeutically effective amount of the electrical stimulus and a second motoneuron excitability after the application.

41. The method of claim 39 or claim 40, wherein the electrical stimulus comprises electrical pulses having an amplitude of about 10 μA to about 100 mA, a width between about 40 μs and about 2 ms, and a frequency of about 10 Hz to about 2000 Hz.

42. The method of any one of claims 39-41, wherein the electrical stimulus is applied for at least 1 hour/day over a period of at least 1 month.

43. The method of any one of claims 39-42, wherein the motoneuron excitability of the subject is computed based on electrical signals produced by the motoneurons while varying the parameters of the electrical stimulus.

44. The method of any one of claims 39-43, wherein increasing excitability of motoneurons impaired by SMA increases joint torques and muscle strength of the subject.

45. A therapeutically effective amount of an electrical stimulus to sensory neurons innervating a body region of the subject with a motor impairment due to SMA for treating SMA in a subject, wherein the electrical stimulus is to be applied with one or more electrodes controlled by a neurostimulator, and wherein application of the electrical stimulus treats the motor impairment due to SMA in the subject.

46. The electrical stimulus for use of claim 45, wherein applying the electrical stimulus increases the firing rate probability of spinal motoneurons innervating the body region of the subject with the motor impairment due to SMA.

47. The electrical stimulus for use of claim 45 or claim 46, wherein the stimulation is applied at or below a motor threshold such that the stimulation does not directly elicit movement and/or muscle activity of the body region of the subject with the motor impairment due to SMA.

48. The electrical stimulus for use of claims 45-47, wherein the electrical stimulus comprises electrical pulses having an amplitude of about 10 μA to about 100 mA, a width between about 40 μs and about 2 ms, and a frequency of about 10 Hz to about 2000 Hz.

49. The electrical stimulus for use of claims 45-48, wherein the electrical stimulus comprises electrical pulses having an amplitude of about 10 μA to about 10 mA, a width between about 40 μs and about 2 ms, and a frequency of about 10 Hz to about 1000 Hz.

50. The electrical stimulus for use of claims 45-49, wherein the electrical stimulus comprises electrical pulses having an amplitude of about 100 μA to about 10 mA, a width between about 40 μs and about 500 μs, and a frequency of about 10 Hz to about 1000 Hz.

51. The electrical stimulus for use of any one of the prior claims, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

52. The electrical stimulus for use of claim 51, wherein the stimulation pattern is a series of 3 pulses separated by inter-pulse intervals of about 5 ms, wherein the series is repeated at a frequency of about 30 to about 100 Hz, and wherein the pulse width is about 200 μs.

53. The electrical stimulus for use of any one of claims 48-52, wherein the pulses are cathodic-first biphasic or monophasic charge balanced pulses.

54. The electrical stimulus for use of any one of the prior claims, wherein the electrical stimulus is applied for at least 2 hours/day over a period of at least 6 months.

55. The electrical stimulus for use of any one of claims 45-53, wherein the electrical stimulus is applied for at least 1 hour/day over a period of at least 1 month.

56. The electrical stimulus for use of any one of the prior claims, wherein the one or more electrodes are contained within an array of independently controllable electrodes implanted in the subject.

57. The electrical stimulus for use of claim 56, wherein the electrode array is a multi-electrode paddle array.

58. The electrical stimulus for use of any one of the prior claims, wherein the one or more electrodes are implanted epidurally at the spinal cord of the subject.

59. The electrical stimulus for use of any one of claims 45-58, wherein the one or more electrodes are implanted at the dorsal rootlets of the one or more sensory neurons innervating the body region with the motor impairment of the subject.

60. The electrical stimulus for use of any one of claims 45-58, wherein the one or more electrodes are implanted at the dorsolateral aspect of the spinal cord adjacent to the dorsal roots of the one or more sensory neurons innervating the body region with the motor impairment of the subject.

61. The electrical stimulus for use of any one of claims 45-57, wherein the one or more electrodes are implanted at the dorsal root ganglia of the one or more sensory neurons innervating the body region with the motor impairment of the subject.

62. The electrical stimulus for use of any one of claims 45-56, wherein the one or more electrodes are contained in a cuff that surrounds a peripheral nerve containing the sensory neurons innervating the body region of the subject with the motor impairment due to SMA.

63. The electrical stimulus for use of any one of claims 45-56, wherein the one or more electrodes penetrate a peripheral nerve or dorsal root ganglion containing the sensory neurons innervating the body region of the subject with the motor impairment due to SMA.

64. The electrical stimulus for use of any one of the prior claims, wherein the body region of the subject is selected from at least one of lower back, hip, leg, ankle, and foot; and the one or more electrodes are implanted at the dorsolateral aspect of the spinal cord and span one or more of the T11-S1 nerve roots.

65. The electrical stimulus for use of any one of the prior claims, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

66. The electrical stimulus for use of any one of the prior claims, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

67. The electrical stimulus for use of any one of the prior claims, wherein the neurostimulator is an external or implanted pulse generator.

68. The electrical stimulus for use of any one of the prior claims, wherein the neurostimulator is implanted in the subject.

69. The electrical stimulus for use of any one of the prior claims, wherein the subject with SMA is selected for treatment.

70. The electrical stimulus for use of any one of the prior claims, wherein the motor impairment comprises partial or complete paralysis, loss of dexterity, loss of muscle strength, and/or uncontrollable muscle tone.

71. The electrical stimulus for use of any one of the prior claims, wherein the locations of the one or more electrodes are chosen based on the body region of the subject with the motor impairment due to SMA.

72. The electrical stimulus for use of claim 71, wherein the locations of the one or more electrodes are determined by:
stimulating a plurality of contact points in the subject to target at least one muscle within the body region of the subject with the motor impairment due to SMA;
measuring the electrical activity associated with said muscle in response to the stimulation; and
identifying one or more contact points in the plurality of contact points as locations of the electrodes based on said electrical activity.

73. The electrical stimulus for use of any one of the prior claims, wherein at least one of the one or more electrodes is placed transcutaneously.

74. The electrical stimulus for use of any one of the prior claims, wherein at least one of the one or more electrodes is implanted.

75. Use of one or more electrodes controlled by a neurostimulator in the manufacture of a medicament for treating SMA in a subject, wherein the one or more electrodes controlled by the neurostimulator are configured to apply a therapeutically effective amount of an electrical stimulus to sensory neurons innervating a body region of the subject with a motor impairment due to SMA.

76. The use of claim 75, wherein applying the electrical stimulus increases the firing rate probability of spinal motoneurons innervating the body region of the subject with the motor impairment due to SMA.

77. The use of claim 75 or claim 76, wherein the stimulation is applied at or below a motor threshold such that the stimulation does not directly elicit movement and/or muscle activity of the body region of the subject with the motor impairment due to SMA.

78. The use of claims 75-77, wherein the electrical stimulus comprises electrical pulses having an amplitude of about 10 μA to about 100 mA, a width between about 40 μs and about 2 ms, and a frequency of about 10 Hz to about 2000 Hz.

79. The use of claims 75-78, wherein the electrical stimulus comprises electrical pulses having an amplitude of about 10 μA to about 10 mA, a width between about 40 μs and about 2 ms, and a frequency of about 10 Hz to about 1000 Hz.

80. The use of claims 75-79, wherein the electrical stimulus comprises electrical pulses having an amplitude of about 100 μA to about 10 mA, a width between about 40 μs and about 500 μs, and a frequency of about 10 Hz to about 1000 Hz.

81. The use of any one of the prior claims, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

82. The use of claim 81, wherein the stimulation pattern is a series of 3 pulses separated by inter-pulse intervals of about 5 ms, wherein the series is repeated at a frequency of about 30 to about 100 Hz, and wherein the pulse width is about 200 μs.

83. The use of any one of claims 78-82, wherein the pulses are cathodic-first biphasic or monophasic charge balanced pulses.

84. The use of any one of the prior claims, wherein the electrical stimulus is applied for at least 2 hours/day over a period of at least 6 months.

85. The use of any one of claims 75-83, wherein the electrical stimulus is applied for at least 1 hour/day over a period of at least 1 month.

86. The use of any one of the prior claims, wherein the one or more electrodes are contained within an array of independently controllable electrodes implanted in the subject.

87. The use of claim 86, wherein the electrode array is a multi-electrode paddle array.

88. The use of any one of the prior claims, wherein the one or more electrodes are implanted epidurally at the spinal cord of the subject.

89. The use of any one of claims 75-88, wherein the one or more electrodes are implanted at the dorsal rootlets of the one or more sensory neurons innervating the body region with the motor impairment of the subject.

90. The use of any one of claims 75-88, wherein the one or more electrodes are implanted at the dorsolateral aspect of the spinal cord adjacent to the dorsal roots of the one or more sensory neurons innervating the body region with the motor impairment of the subject.

91. The use of any one of claims 75-87, wherein the one or more electrodes are implanted at the dorsal root ganglia of the one or more sensory neurons innervating the body region with the motor impairment of the subject.

92. The use of any one of claims 75-86, wherein the one or more electrodes are contained in a cuff that surrounds a peripheral nerve containing the sensory neurons innervating the body region of the subject with the motor impairment due to SMA.

93. The use of any one of claims 75-86, wherein the one or more electrodes penetrate a peripheral nerve or dorsal root ganglion containing the sensory neurons innervating the body region of the subject with the motor impairment due to SMA.

94. The use of any one of the prior claims, wherein the body region of the subject is selected from at least one of lower back, hip, leg, ankle, and foot; and the one or more electrodes are implanted at the dorsolateral aspect of the spinal cord and span one or more of the T11-S1 nerve roots.

95. The use of any one of the prior claims, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

96. The use of any one of the prior claims, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

97. The use of any one of the prior claims, wherein the neurostimulator is an external or implanted pulse generator.

98. The use of any one of the prior claims, wherein the neurostimulator is implanted in the subject.

99. The use of any one of the prior claims, wherein the subject with SMA is selected for treatment.

100. The use of any one of the prior claims, wherein the motor impairment comprises partial or complete paralysis, loss of dexterity, loss of muscle strength, and/or uncontrollable muscle tone.

101. The use of any one of the prior claims, wherein the locations of the one or more electrodes are chosen based on the body region of the subject with the motor impairment due to SMA.

102. The use of claim 101, wherein the locations of the one or more electrodes are determined by:
stimulating a plurality of contact points in the subject to target at least one muscle within the body region of the subject with the motor impairment due to SMA;
measuring the electrical activity associated with said muscle in response to the stimulation; and
identifying one or more contact points in the plurality of contact points as locations of the electrodes based on said electrical activity.

103. The use of any one of the prior claims, wherein at least one of the one or more electrodes is placed transcutaneously.

104. The use of any one of the prior claims, wherein at least one of the one or more electrodes is implanted.

105. Use of one or more electrodes controlled by a neurostimulator in the manufacture of a medicament for treating SMA in a subject, wherein an electrical stimulus is to be applied using the one or more electrodes controlled by the neurostimulator to sensory neurons innervating a body region of the subject with a motor impairment due to SMA.

106. The use of claim 105, wherein applying the electrical stimulus increases the firing rate probability of spinal motoneurons innervating the body region of the subject with the motor impairment due to SMA.

107. The use of claim 105 or claim 106, wherein the stimulation is applied at or below a motor threshold such that the stimulation does not directly elicit movement and/or muscle activity of the body region of the subject with the motor impairment due to SMA.

108. The use of claims 105-107, wherein the electrical stimulus comprises electrical pulses having an amplitude of about 10 μA to about 100 mA, a width between about 40 μs and about 2 ms, and a frequency of about 10 Hz to about 2000 Hz.

109. The use of claims 105-108, wherein the electrical stimulus comprises electrical pulses having an amplitude of about 10 μA to about 10 mA, a width between about 40 μs and about 2 ms, and a frequency of about 10 Hz to about 1000 Hz.

110. The use of claims 105-109, wherein the electrical stimulus comprises electrical pulses having an amplitude of about 100 μA to about 10 mA, a width between about 40 μs and about 500 μs, and a frequency of about 10 Hz to about 1000 Hz.

111. The use of any one of the prior claims, wherein the electrical stimulus comprises a stimulation pattern of a series of 2 to 5 pulses separated by inter-pulse intervals of about 3 ms to about 10 ms and wherein the series is repeated at a frequency of about 10 Hz to about 100 Hz.

112. The use of claim 111, wherein the stimulation pattern is a series of 3 pulses separated by inter-pulse intervals of about 5 ms, wherein the series is repeated at a frequency of about 30 to about 100 Hz, and wherein the pulse width is about 200 μs.

113. The use of any one of claims 108-112, wherein the pulses are cathodic-first biphasic or monophasic charge balanced pulses.

114. The use of any one of the prior claims, wherein the electrical stimulus is applied for at least 2 hours/day over a period of at least 6 months.

115. The use of any one of claims 105-113, wherein the electrical stimulus is applied for at least 1 hour/day over a period of at least 1 month.

116. The use of any one of the prior claims, wherein the one or more electrodes are contained within an array of independently controllable electrodes implanted in the subject.

117. The use of claim 116, wherein the electrode array is a multi-electrode paddle array.

118. The use of any one of the prior claims, wherein the one or more electrodes are implanted epidurally at the spinal cord of the subject.

119. The use of any one of claims 105-118, wherein the one or more electrodes are implanted at the dorsal rootlets of the one or more sensory neurons innervating the body region with the motor impairment of the subject.

120. The use of any one of claims 105-118, wherein the one or more electrodes are implanted at the dorsolateral aspect of the spinal cord adjacent to the dorsal roots of the one or more sensory neurons innervating the body region with the motor impairment of the subject.

121. The use of any one of claims 105-117, wherein the one or more electrodes are implanted at the dorsal root ganglia of the one or more sensory neurons innervating the body region with the motor impairment of the subject.

122. The use of any one of claims 105-116, wherein the one or more electrodes are contained in a cuff that surrounds a peripheral nerve containing the sensory neurons innervating the body region of the subject with the motor impairment due to SMA.

123. The use of any one of claims 105-116, wherein the one or more electrodes penetrate a peripheral nerve or dorsal root ganglion containing the sensory neurons innervating the body region of the subject with the motor impairment due to SMA.

124. The use of any one of the prior claims, wherein the body region of the subject is selected from at least one of lower back, hip, leg, ankle, and foot; and the one or more electrodes are implanted at the dorsolateral aspect of the spinal cord and span one or more of the T11-S1 nerve roots.

125. The use of any one of the prior claims, wherein the body region of the subject is selected from at least one of upper arm, shoulder, arm, hand, and respiratory muscles; and the one or more electrodes are implanted at the dorsolateral aspect of the spinal cord and span one or more of the C3-T2 nerve roots.

126. The use of any one of the prior claims, wherein the body region of the subject is selected from at least one of the chest, chest wall, abdomen, upper back, and middle back; and the one or more electrodes are implanted at the dorsolateral aspect of the spinal cord and span one or more of the T3-T10 nerve roots.

127. The use of any one of the prior claims, wherein the neurostimulator is an external or implanted pulse generator.

128. The use of any one of the prior claims, wherein the neurostimulator is implanted in the subject.

129. The use of any one of the prior claims, wherein the subject with SMA is selected for treatment.

130. The use of any one of the prior claims, wherein the motor impairment comprises partial or complete paralysis, loss of dexterity, loss of muscle strength, and/or uncontrollable muscle tone.

131. The use of any one of the prior claims, wherein the locations of the one or more electrodes are chosen based on the body region of the subject with the motor impairment due to SMA.

132. The use of claim 131, wherein the locations of the one or more electrodes are determined by:
stimulating a plurality of contact points in the subject to target at least one muscle within the body region of the subject with the motor impairment due to SMA;
measuring the electrical activity associated with said muscle in response to the stimulation; and
identifying one or more contact points in the plurality of contact points as locations of the electrodes based on said electrical activity.

133. The use of any one of the prior claims, wherein at least one of the one or more electrodes is placed transcutaneously.

134. The use of any one of the prior claims, wherein at least one of the one or more electrodes is implanted.
